(19) United States
(12) Patent Application Publication
(10) Pub. No.: US 2026/0062698 A1
(43) Pub. Date: Mar. 5, 2026

(54) HIGH DRUG-LOADING RATIO APTAMER-DRUG CONJUGATES AND ITS APPLICATION

(71) Applicant: Hangzhou Institute of Medicine Chinese Academy of Science, Hangzhou, Zhejiang (CN)

(72) Inventors: Weihong TAN, Hangzhou, Zhejiang (CN); Jiaxuan HE, Hangzhou, Zhejiang (CN); Xiangsheng LIU, Hangzhou, Zhejiang (CN); Minhui SU, Hangzhou, Zhejiang (CN); Yanlan LIU, Hangzhou, Zhejiang (CN); Sitao XIE, Hangzhou, Zhejiang (CN); Ting FU, Hangzhou, Zhejiang (CN)

(21) Appl. No.: 19/069,719

(22) Filed: Mar. 4, 2025

(30) Foreign Application Priority Data
Aug. 30, 2024 (CN) 202411216202X

(51) Int. Cl. C12N 15/113; A61K 31/40; A61K 31/437; A61K 47/54

(52) U.S. Cl. CPC C12N 15/113; A61K 31/40; A61K 31/437; A61K 47/549; C12N 2310/16; C12N 2310/314; C12N 2320/32

(57) ABSTRACT

The invention provides a high drug-loading ratio aptamer-drug conjugates (ApDC) and its applications. By using a linker with a dendritic structure and combining solid-phase synthesis with efficient click chemistry reactions, the invention enables the design and efficient synthesis of high drug-loading ratio ApDC. These drugs demonstrate excellent tumor-inhibiting activity in antitumor applications. Moreover, different drug-loading ratios can be designed to achieve higher efficacy for different drugs, resulting in ApDC with enhanced therapeutic effects. The invention exhibits outstanding antitumor effects against malignant tumors such as lung cancer, colorectal cancer, breast cancer, and ovarian cancer, with an inhibition efficiency exceeding 90%.

### CROSS-REFERENCE TO RELATED APPLICATIONS

This application claims priority to Chinese Patent Application, Application No.: 202411216202X, filed on Aug. 30, 2024, and all disclosures of this application are incorporated by reference in their entirety as a part of this application.

### REFERENCE TO AN ELECTRONIC SEQUENCE LISTING

The contents of the electronic sequence listing (sequence listing.xml; Size: 2,943 bytes; and Date of Creation: Mar. 3, 2025) is herein incorporated by reference in its entirety.

### BACKGROUND OF THE INVENTION

### Field of the Invention

The present invention belongs to the field of biotechnology, specifically relating to a high drug-loading ratio aptamer-drug conjugates and its applications.

### Description of the Related Art

Targeted drug conjugates, especially antibody-drug conjugates (ADCs), have driven enthusiasm for mergers and acquisitions among companies due to their clinical outcomes and potential commercial value, attracting widespread attention in the industry. Technological advancements have also led to a convergence and clash of new and old concepts in targeted drug conjugates, presenting challenges to the current ideas and technologies surrounding targeted drug conjugates.

Aptamer-drug conjugates (ApDC) are highly promising targeted drug conjugates due to their ease of chemical synthesis and modification. Currently, the vast majority of ApDC primarily load a single drug. High drug-loading ratio ApDC primarily involve the conjugation of high drug-loading ratio drug precursors with aptamers. However, the synthesis of high drug-loading ratio drug precursors is complex, and their poor water solubility results in low conjugation efficiency with aptamers. Therefore, there is an urgent need to find a high drug-loading ratio ApDC that can precisely control the drug-loading ratio to achieve efficient conjugation while significantly enhancing efficacy to meet clinical demands.

### BRIEF SUMMARY OF THE INVENTION

To overcome the problems, the present invention provides a high drug-loading ratio ApDC and its applications. By using a linker with a dendritic structure and integrating solid-phase synthesis with efficient click chemistry reactions, the invention enables the design and efficient synthesis of high drug-loading ratio ApDC. These drugs demonstrate high antitumor activity in applications, and by designing ApDC with different drug-loading ratios for various drugs, the efficacy can be enhanced. The invention shows excellent antitumor effects against malignant tumors such as colorectal cancer, breast cancer, lung cancer, and ovarian cancer, with an inhibition efficiency exceeding 90%.

As one of the aspects, the invention provides a high drug-loading ApDC, which is prepared by conjugating a multi-conjugation site aptamer with a drug. The multi-conjugation site aptamer includes an aptamer and dendritic phosphoramidite monomers, the 3′ and/or 5′ end of the aptamer being connected to the dendritic phosphoramidite monomer. The end of the aptamer is connected with the dendritic phosphoramidite monomer with one or more reactive groups that are selected from thiol, amino, DBCO, azide, or maleimide. The dendritic phosphoramidite monomer includes any one or more of Phosphoramidite 1, Phosphoramidite 2, and Phosphoramidite 3, with the structures of Phosphoramidite 1, Phosphoramidite 2, and Phosphoramidite 3 being as follows:

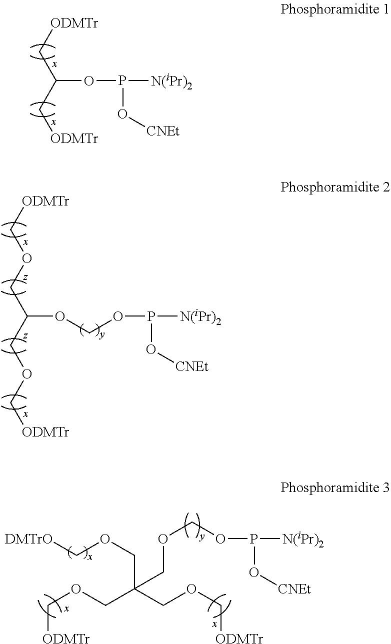

In some embodiments, and x is an integer from 1 to n; y is an integer from 1 to n; z is an integer from 1 to n; DMTr is a 4,4′-dimethoxytrityl protecting group, ⁱPr is isopropyl, and CNEt is cyanoethyl.

Traditional drug-loading ratio ApDC have low conjugation efficiency, making it difficult to achieve precise and efficient conjugation with drugs. This invention utilizes a linker with a dendritic structure and employs solid-phase synthesis technology to create ApDC with a precisely controllable drug-loading ratio. By using phosphodiester bonds as the backbone of the linker, the water solubility of the drug is improved, thereby increasing the drug conjugation efficiency. The IC50 of the high drug-loading ratio ApDC is significantly reduced compared to single-loaded ApDC (for example, for ovarian cancer SKOV-3 cells, the IC50 is reduced by more than eight times compared to single-loaded ApDC).

The aptamer-drug conjugate provided by this invention with a precisely controllable drug-loading ratio specifically includes an aptamer portion, an intermediate dendritic linker portion, and a cytotoxic drug portion. The aptamer portion comprises an aptamer that can recognize tumor cell-related targets, the linker portion is based on nucleic acid solid-phase synthesis and features bifurcated and trifurcated structures, while the cytotoxic drug portion comprises commonly used auristatin-class microtubule inhibitors or their derivatives, exatecan or its derivatives. The aptamer-drug conjugate with a precisely controllable drug-loading ratio can be used to construct ApDC with 2, 3, 4, 6, and 8 drugs, and can be applied for the treatment of malignant tumors.

The dendritic phosphoramidite monomers include bifurcated monomers (Phosphoramidite 1 or Phosphoramidite 2), trifurcated monomers (Phosphoramidite 3), or combinations thereof, and the solid-phase synthesis method is employed to construct multi-conjugation site aptamers.

The method for solid-phase synthesis of multi-conjugation site aptamers is as follows: The aptamer is synthesized using a solid-phase synthesizer, and after completing the solid-phase synthesis of the aptamer, the preparation of the di-conjugation site aptamer is carried out according to the method of solid-phase synthesis.

The specific operation is as follows:
(1) Prepare a 3% trichloroacetic acid/dichloromethane solution to remove the 4,4′-dimethoxytrityl (DMTr) group from the hydroxyl group at the 5′ end of the nucleic acid chain. After the reaction is complete, wash away the residual reagents with anhydrous acetonitrile.
(2) Prepare a solution of bifurcated phosphoramidite monomer, dissolving it in anhydrous acetonitrile to achieve a concentration of 0.1 M. The activating agent chosen is tetrazole, which is dissolved in anhydrous acetonitrile to a concentration of 0.5 M. Mix the monomer and tetrazole in a volume ratio of 2:3 and conduct the conjugation reaction. After the reaction is complete, wash away the residual reagents with anhydrous acetonitrile.
(3) Prepare a 0.05 M iodine solution, using a solvent consisting of a 1:3 volume ratio of pyridine to tetrahydrofuran. This is used to oxidize the 3-valent phosphate bond formed between bases to a 5-valent phosphate bond. After completion of the reaction, wash away the residual reagents with anhydrous acetonitrile. After completing the above steps, a product containing two hydroxyl sites is obtained. The following step involves the conjugation of thiols according to solid-phase synthesis methods.
(4) Repeat the conjugation reaction of step (2) once more. (5) Prepare a solution of thiol phosphoramidite monomer, dissolving it in anhydrous acetonitrile to achieve a concentration of 0.1 M. The activating agent chosen is tetrazole, which is dissolved in anhydrous acetonitrile to a concentration of 0.5 M. Mix the monomer and tetrazole in a volume ratio of 2:3 and conduct the conjugation reaction. After the reaction is complete, wash away the residual reagents with anhydrous acetonitrile.
(6) Repeat the conjugation reaction of step (2) once more.
(7) Repeat the oxidization reaction of step (3) once more. After completing the above steps, a product with protected thiol at the two conjugation sites is obtained. The aptamer is then cleaved from the solid-phase support, and TCEP is used to reduce the disulfide bonds, followed by purification to obtain the aptamer with two conjugation sites.
   Preparation process of aptamers with di-conjugation site as shown below.

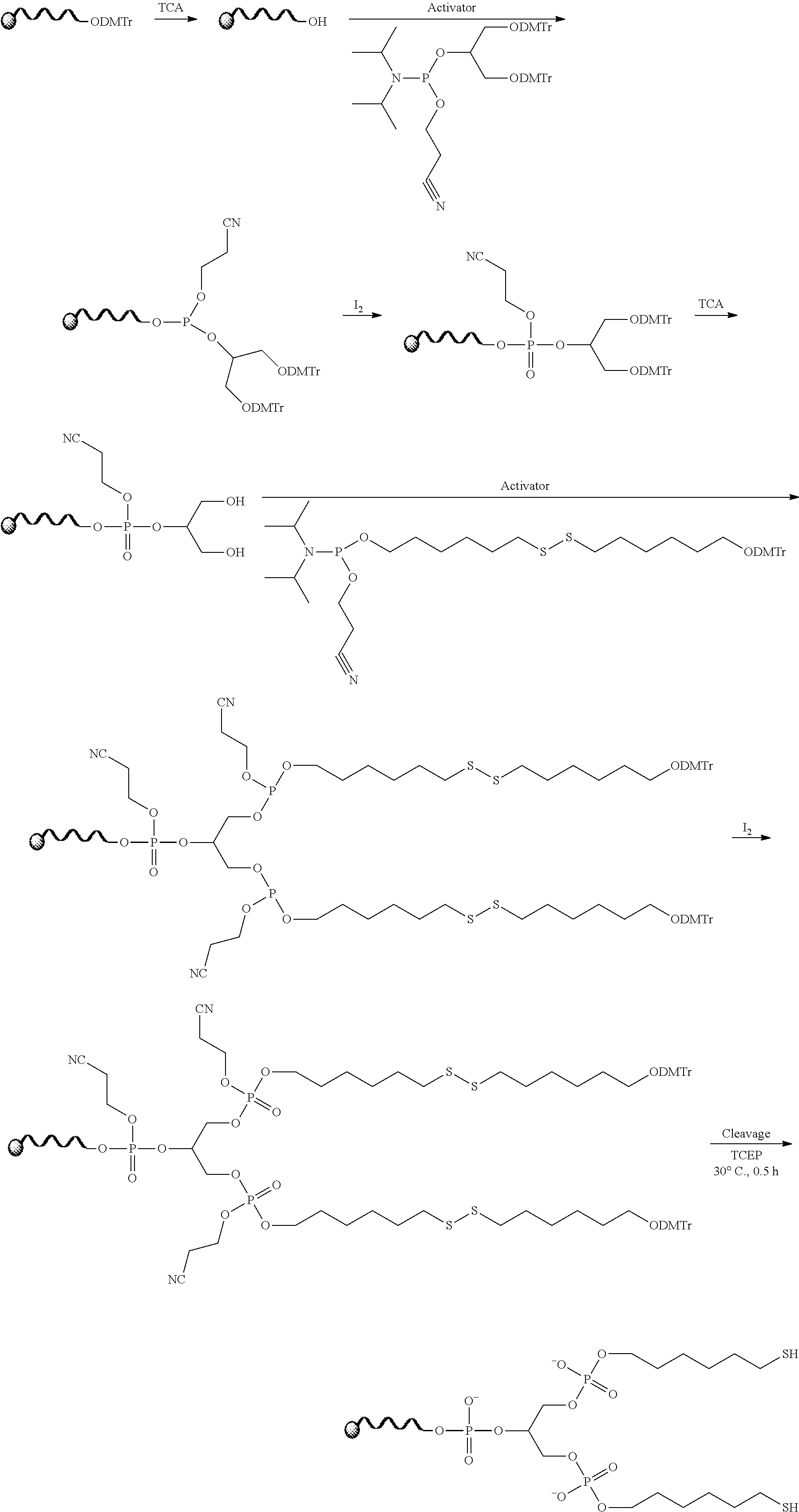

The preparation of aptamers with four or more conjugation sites is similar to that of the di-conjugation site aptamer. By conjugating the bifurcated linker twice, a tetra-conjugation site aptamer is obtained. By conjugating the bifurcated linker three times, an octa-conjugation site aptamer is obtained. By conjugating the bifurcated linker once and the trifurcated linker once, a hexa-conjugation site aptamer is obtained, and so on. Preparation process of aptamers with tetra-conjugation site as shown below.

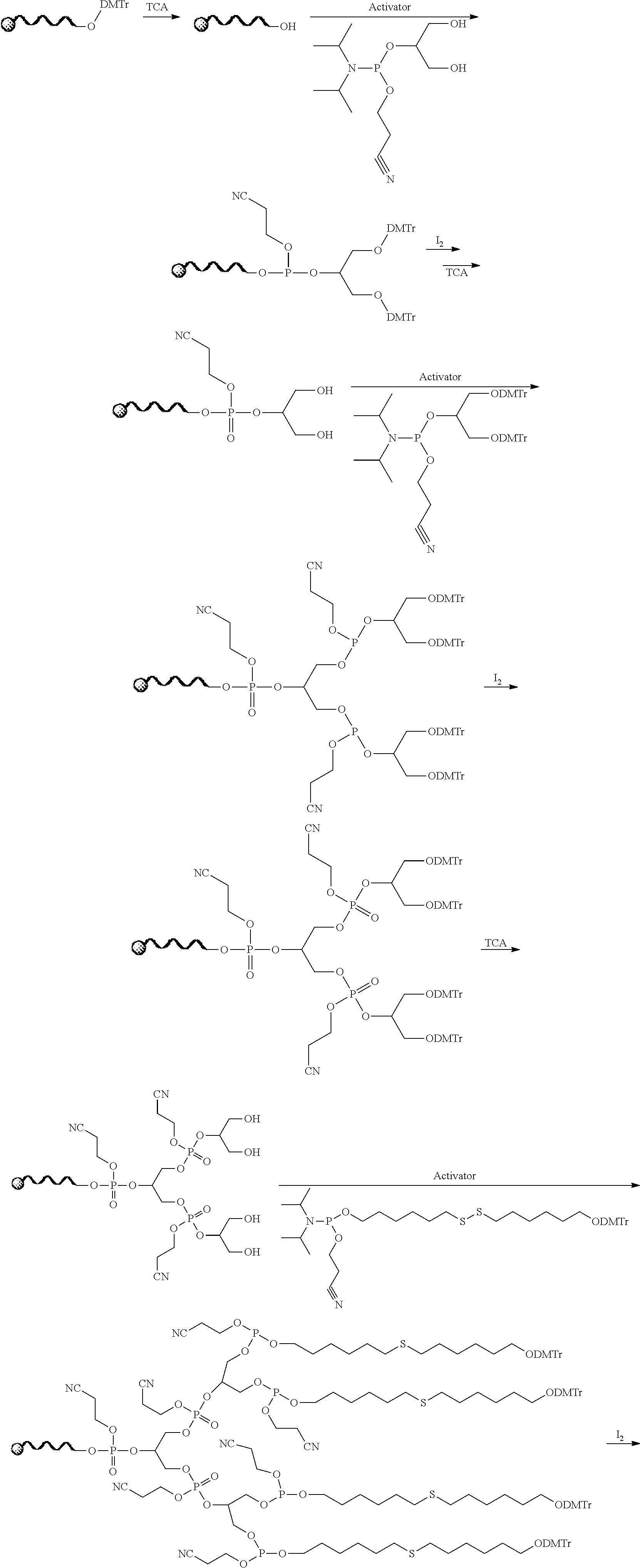

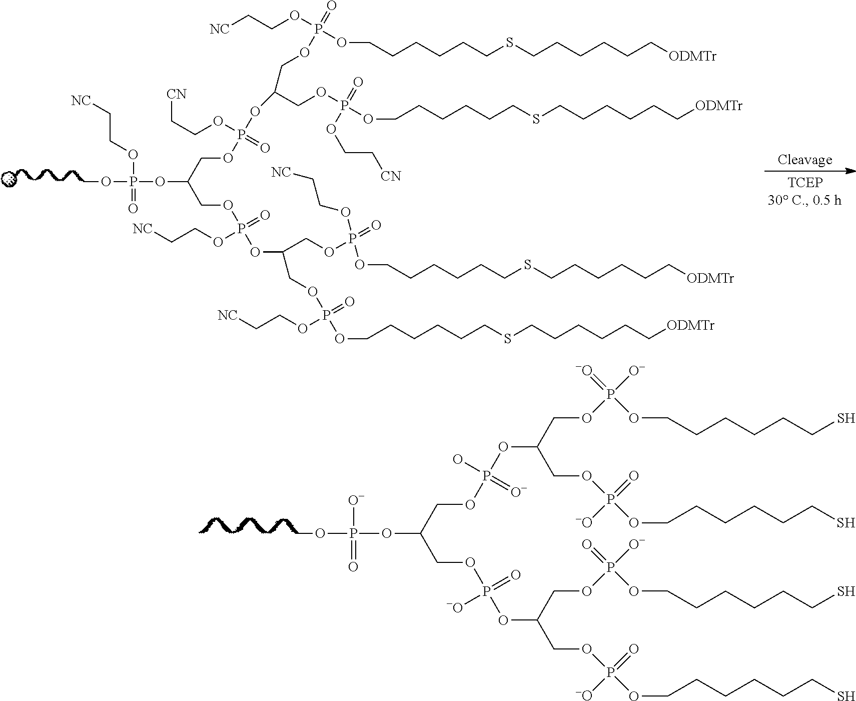

Furthermore, the aptamer targets or conjugate with one or more of sites of the following antigens: CD5, CD19, CD20, CD25, CD37, CD30, CD33, CD45, CAMPATH-1, HLA-DR, CEA, TAG-72, EpCAM, MUC1, MUC15, folate-binding protein, A33, G250, PSMA, ferritin, GD2, GD3, GM2, Leg, CA-125, CA19-9, epidermal growth factor, p185HER2, IL-2 receptor, tenascin, a metalloproteinase, endosialin, vascular endothelial growth factor, avB3, WT1, LMP2, HPV E6, HPV E7, EGFR, EGFRVIII, Her-2/neu, MAGE A3, p53 nonmutant, NY-ESO-1, MelanA/MART1, Ras mutant, gp100, p53 mutant, PR1, bcr-abl, tyrosinase, survivin, PSA, hTERT, a sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, MSLN, PSCA, MAGE AI, MAGE-A3, sLe, CYP1B1, PLAVI, GM3, BORIS, Tn, GloboH, ETV6-AML, NY-BR-1, RGS5, SART3, STn, carbonic anhydrase IX, PAX5, OY-TESL sperm protein 17, LCK, HMWMAA, AKAP-4, 55X2, XAGE 1, B7H3, legumain, Tie 3, Page 4, VEGFR2, MAD-CT-1, PDGFR-B, MAD-CT-2, ROR2, CMET, HER3, EPCAM, CA6, NAPI2B, TROP2, CLDN18.2, FAP, RON, LY6E, FRA, DLL3, PTK7, LIV1, ROR1, Fos-related antigen 1, VEGFR, endoglin, PD-L1, CD204, CD206, CD301, CD71, VTCN1, Nectin-4, and VISTA.

Furthermore, the drug includes any one or more of auristatin E, Dxd, exatecan, or derivatives that have the same or similar core structure as auristatin E, Dxd, or exatecan.

Additionally, the number of conjugation sites of the aptamer is 2, 3, 4, 6, or 8.

Moreover, the linker is conjugated to the 5′ end of the aptamer.

The invention demonstrates, through a comparison of two drugs simultaneously conjugated to both ends of the nucleic acid versus using bifurcated linkers at the 5′ and 3′ ends, that the use of bifurcated linkers significantly enhances the stability of the peptide linker vc in mouse plasma when conjugated to the 5′ end of the aptamer.

Furthermore, when the drug is Dxd, the number of conjugation sites of the aptamer is 4; when the drug is exatecan, the number of conjugation sites of the aptamer is 2 or 4; and when the drug is auristatin E, the number of conjugation sites of the aptamer is 2 or 4.

Research has shown that for different drugs and the treatment of various cancers, it is essential to select an appropriate drug-loading ratio for the ApDC to effectively enhance tumor inhibition activity.

For example, when the drug is Dxd (Deruxtecan), used for the treatment of colorectal cancer, having a 4-ratio drug-loading significantly improves efficacy compared to other drug-loading ratios (including 1, 2, 3, 6, 8, etc.), all while maintaining the same drug content (with consistent drug equivalent concentrations in each structure). This enhancement in efficacy may be attributed to the distribution of drugs in S4D, which optimally balances steric hindrance and synergistic effects between the drugs, making it particularly suitable for the treatment of colorectal cancer and maximizing the therapeutic effect of Dxd. Furthermore, when treating colorectal cancer, the efficacy of S4D exceeds that of exatecan at the same concentration, whereas other formulations of Dxd do not achieve this effect.

In cases where the drug is exatecan or auristatin, used for the treatment of lung cancer or colorectal cancer, there is no significant difference in efficacy when the drug-loading ratio is 2 or 4; however, there is an improvement compared to the situation with single drug-loading. Therefore, it is preferable to use a drug-loading ratio of 2 or 4.

Moreover, when the drug is Dxd, the ApDC is S4D, with the structural formula being:

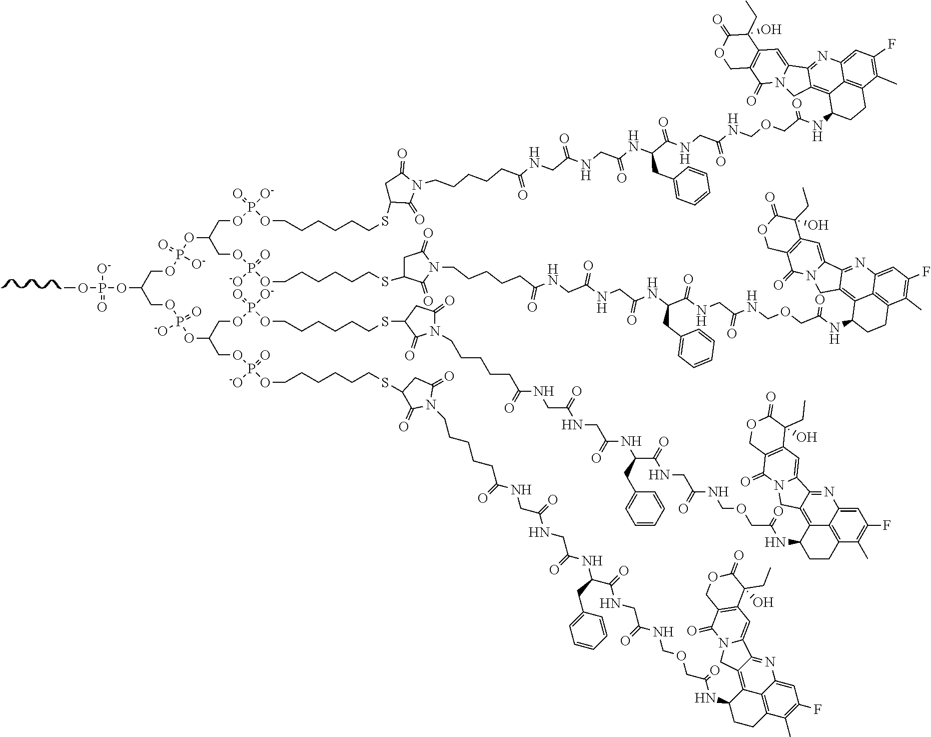

Furthermore, the drug is exatecan, the ApDC is S2E or S4E, with the structural formula of S2E being:

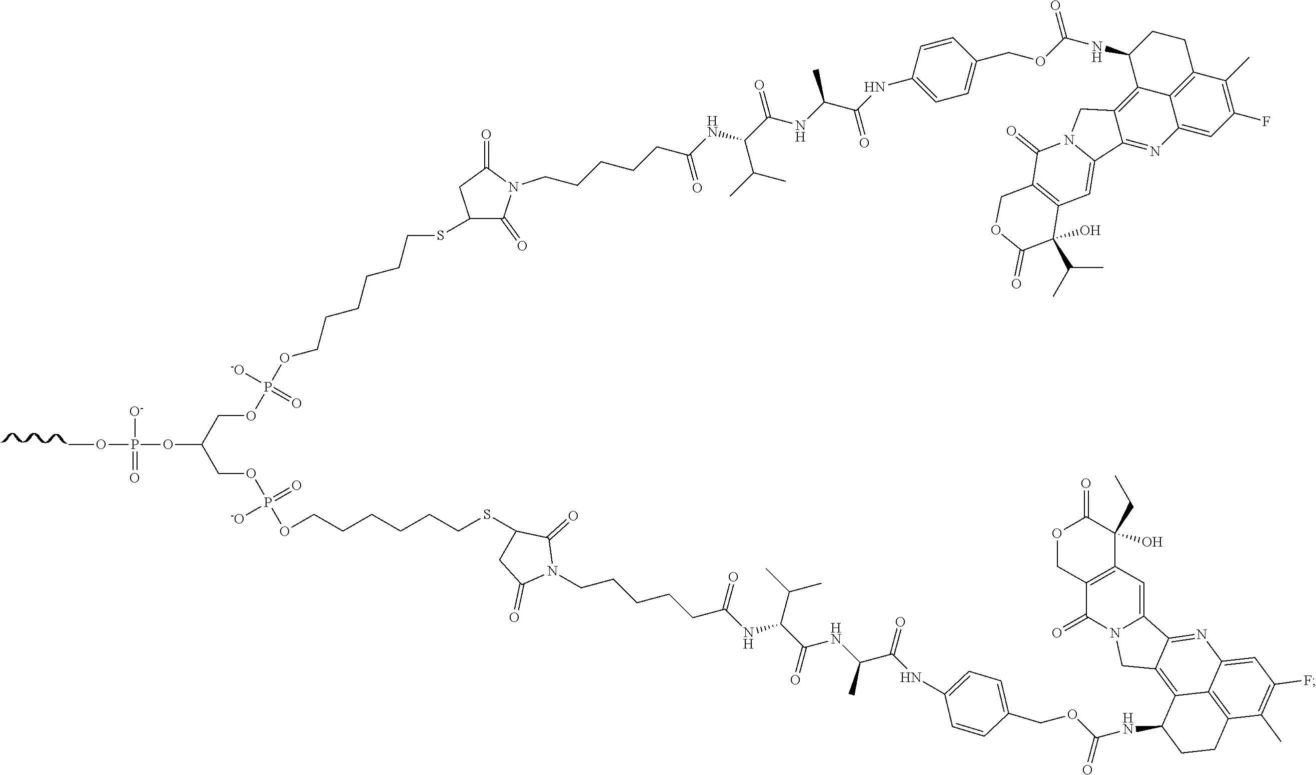

the structural formula of S4E is:

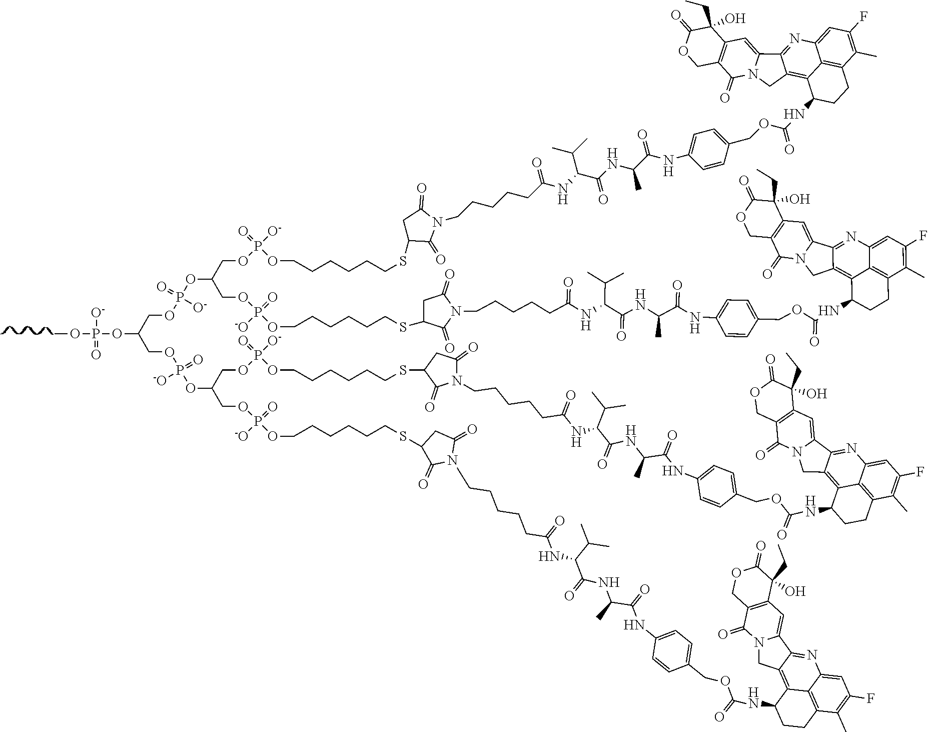

Furthermore, when the drug is auristatin E, the aptamer-drug conjugate is S2M or S4M, with the structural formula of S2M being:

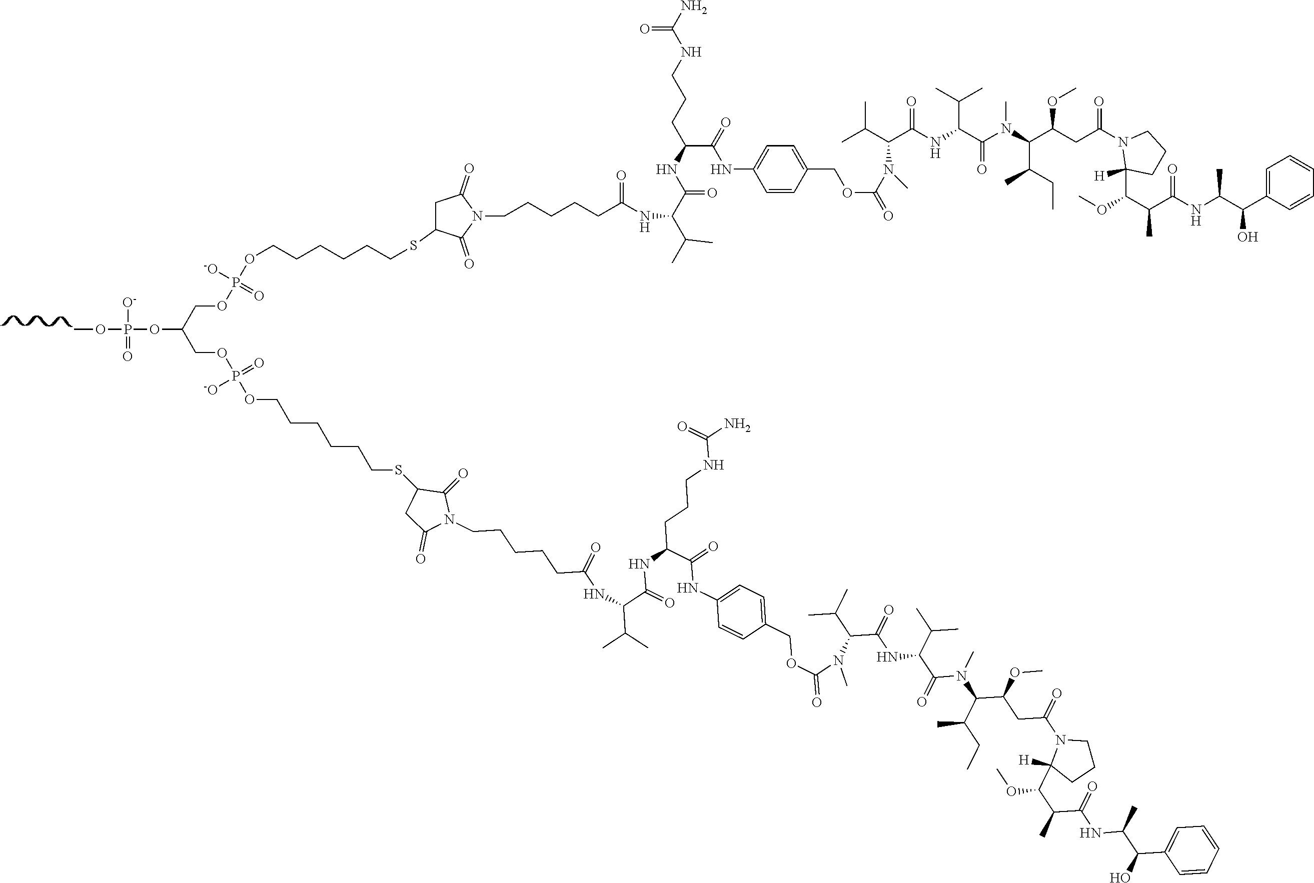

the structural formula of S4M is:

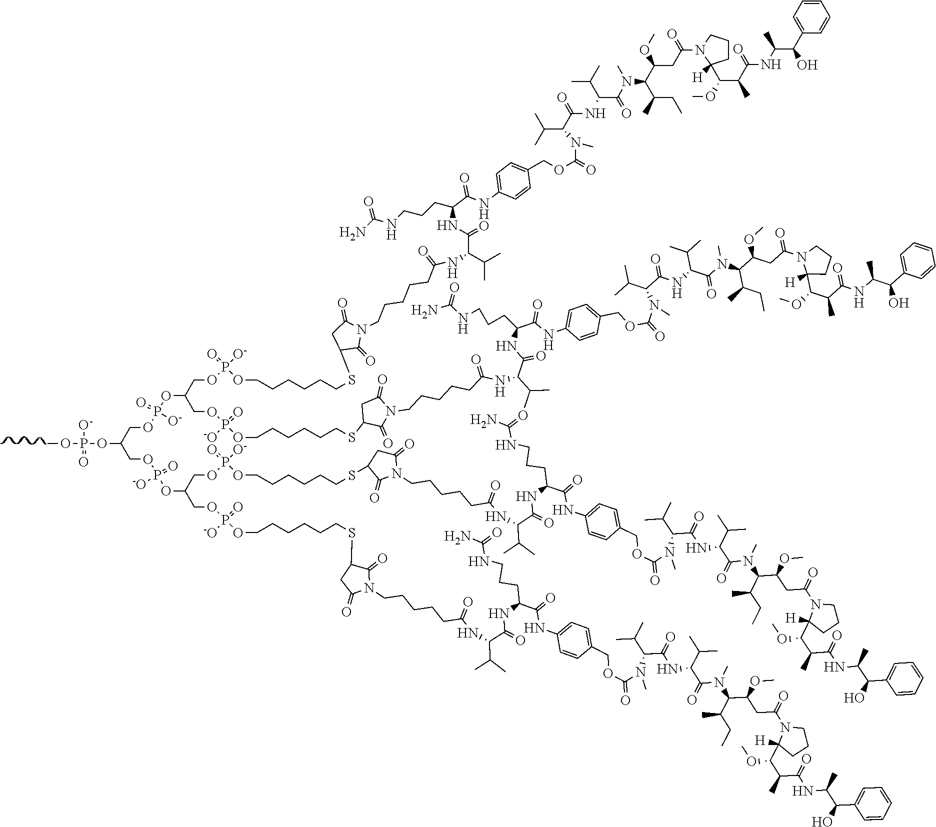

On the other hand, the present invention provides the use of an ApDC for preparing a reagent to enhance the antitumor effect in colorectal cancer, wherein the ApDC is S4D, with the structural formula being:

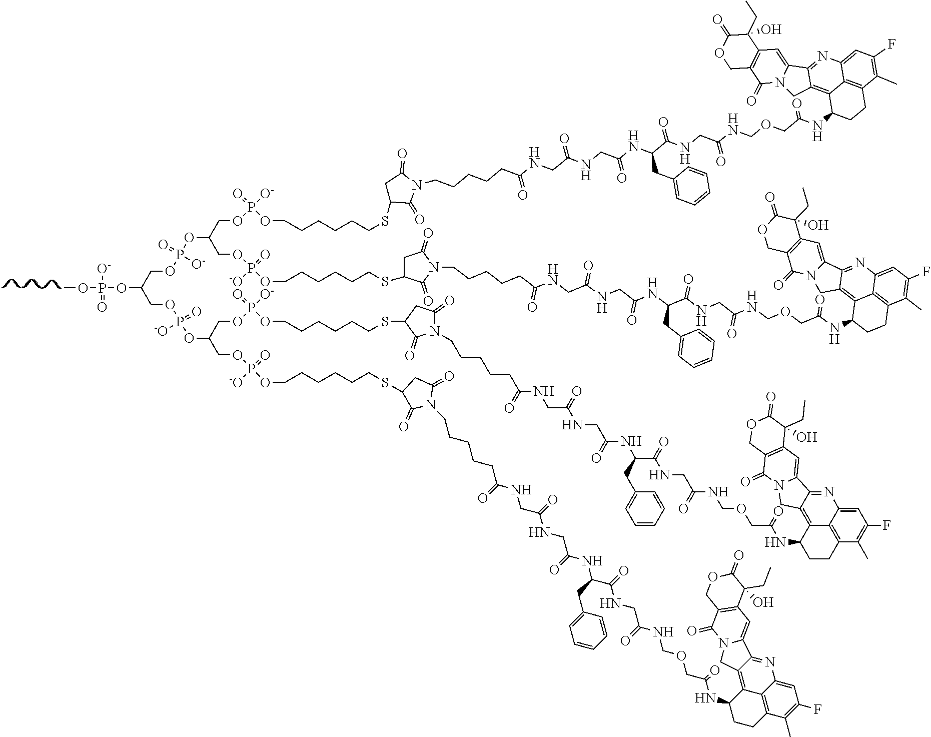

In another aspect, the present invention provides the use of an ApDC for preparing a reagent to enhance the efficacy of the DNA topoisomerase I inhibitor Dxd, thereby exceeding the in vivo antitumor effects of exatecan. The ApDC is S4D, with the structural formula being:

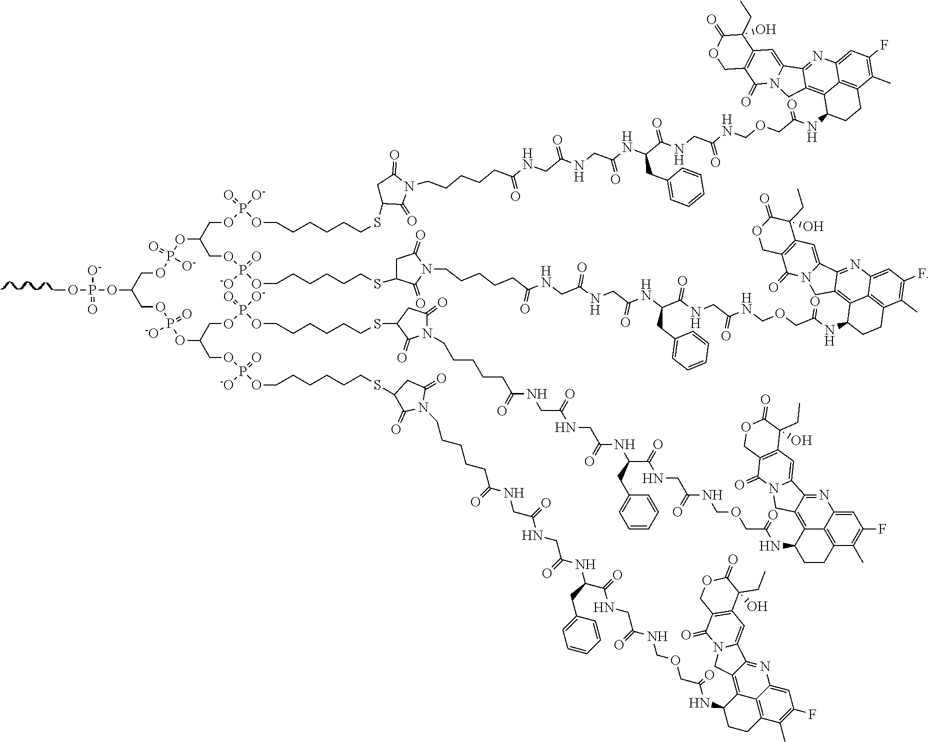

In another aspect, the present invention provides the a method for enhancing the therapeutic effect in malignant tumor cell models, wherein the drug-loading ratio ApDC comprises all of the embodiments as above descripted.

In some embodiments, the malignant tumor cell models include lung cancer, breast cancer, liver cancer, gastric cancer, pancreatic cancer, colorectal cancer, thyroid cancer, prostate cancer, ovarian cancer, neuroblastoma, glioma, other high-expression targeted aptamer target protein cell transplantation models or PDX models.

In another aspect, the present invention provides a method for preparing an ApDC with a precisely controllable drug-loading ratio, which includes: (1) synthesizing multi-conjugation site aptamers based on solid-phase synthesis; (2) efficiently and precisely constructing high drug-loading ratio ApDC.

Furthermore, it also includes research on the antitumor applications of the high drug-loading ratio ApDC.

The multi-conjugation site aptamer uses a mixture of one or more of the dendritic phosphoramidite monomers (Phosphoramidite 1-3), utilizing a nucleic acid solid-phase synthesizer, and incorporates reactive groups such as thiols, amines, DBCO, azides, and maleimides at the ends for drug conjugation.

In some embodiments, the reactive sites of the multi-conjugation site aptamer include one of the reactive groups such as thiols, maleimides, azides, alkynes, amines, or carboxylic acids.

In some embodiments, the separation and purification of the multi-conjugation site aptamer use a combination of methods such as high-performance liquid chromatography, size exclusion chromatography, and gel electrophoresis.

In some embodiments, the separation and purification of the high drug-loading ratio ApDC also use a combination of methods such as high-performance liquid chromatography, size exclusion chromatography, and gel electrophoresis.

In some embodiments, the multi-conjugation site aptamer is efficiently conjugated to drug molecules through reactions such as thiol-maleimide, amino-carboxylic acid, and DBCO-azide. The specific synthesis method is as follows.

In the synthesis of auristatin-class drugs, a solution of the multi-conjugation site aptamer modified with terminal thiol groups (1 equivalent) is introduced into a centrifuge tube, accompanied by a mixed solution of auristatin-class microtubule inhibitors dissolved in acetonitrile/water (1:1) at a concentration ranging from 2 to 6 equivalents, preferably at 3 equivalents. The reaction is conducted under a temperature range of 4-40° C., preferably maintained at 37° C., and is stirred for a duration of 2 to 24 hours, preferably 16 hours. Following the reaction, purification is performed using reverse-phase preparative chromatography, after which freeze-drying is employed to obtain the aptamer-drug conjugate with a high drug loading ratio, resulting in an approximate yield of 70%. The product is then subjected to desalting and subsequently freeze-dried for further applications.”

In the synthesis of exatecan-class drugs, a solution containing the multi-conjugation site aptamer modified with terminal thiol groups (1 equivalent) is added to a centrifuge tube. An aqueous solution of the exatecan-class drug is prepared with an organic phase concentration ranging from 20% to 60% (2-20 equivalents, preferably 12 equivalents). The organic phase may consist of acetonitrile, dimethylformamide, dimethyl sulfoxide, dimethylacetamide, or combinations thereof. The reaction is maintained at a temperature of 4-40° C., preferably at 37° C., and stirred for a duration of 1 to 24 hours, preferably for 16 hours. Purification is performed using reverse-phase preparative chromatography, followed by freeze-drying to yield the multi-loaded conjugated drug with an approximate yield of 60%. Subsequently, desalting is conducted, and the product is freeze-dried for future applications.

In some embodiments, the high drug-loading ratio aptamer-drug conjugate is used for experiments involving malignant cell binding and proliferation inhibition, such as: Inoculating human ovarian cancer SKOV-3 cells into a 96-well plate (4000 cells per well) and culturing for 24 hours at 37° C. in a 5% carbon dioxide environment. After that, add a culture medium containing ApDC SD, S2D, and S4D at the same drug concentration but with different drug-loading ratios. After culturing for 24 hours at 37° C. in a 5% carbon dioxide environment, discard the drug-containing culture medium and replace it with drug-free culture medium for an additional 108 hours of continued cultivation. After a total of 120 hours, use the MTS assay to measure the inhibitory effect of the drugs.

In some embodiments, the high drug-loading ratio ApDC is used in methods for inhibiting malignant tumor xenograft model experiments, such as: The mouse model uses female BALB/c nude mice with 5 million human colon cancer HT29 cells injected subcutaneously into the right hind limb. After 15 days, when the tumor volume reaches approximately 50-150 mm³, the mice are randomly divided into 2 groups. The two experimental groups receive treatment every four days, consisting of either a tail vein injection of saline (control) or the ApDC S4M with a drug-loading ratio of 4, at a dose of 0.125 μmol/kg ApDC or 0.5 mg/kg of MMAE. The mice's Body weight and tumor length (a) and width (b) were recorded every two days. The tumor volume (V) is calculated using the formula: V=(a×b²)/2. The experimental endpoint is defined by a tumor volume exceeding 1000 mm³ or a weight loss exceeding 15%, at which point euthanasia is performed on the mice to terminate the experiment.

The methods involved in the steps of this invention, including reverse-phase preparative chromatography, desalting, seeding plates, tail vein administration, flow cytometry experiments, and cell inoculation, are all conventional methods in the field. Skilled personnel in the field, using their common knowledge along with the information provided in the invention, can determine these methods.

The present invention has the following beneficial effects:
1. The precise and controllable construction of high drug-loading ratio ApDC is achieved by introducing multiple reactive groups at the ends of nucleic acid molecules using dendritic monomers in solid-phase synthesis, thereby enabling the conjugation with drug molecules and constructing ApDC capable of loading multiple drugs.
2. The constructed high drug-loading ratio ApDC are used for the treatment of tumor models that highly express aptamer targets, resulting in significantly reduced cytotoxicity IC50 compared to single-loaded ApDC, and a marked improvement in tumor suppression effects in animal models.
3. It has been found that when the drug is Dxd and used for treating colorectal cancer, having 4-ratio drug-loading maximizes the efficacy of Dxd compared to other ratio drug-loading. Moreover, in the treatment of colorectal cancer, the efficacy of S4D exceeds that of the first-line clinical chemotherapy drug exatecan (EXA) at the same concentration, while other formulations of Dxd do not achieve this effect.
4. It has been found that when a bifurcated linker is used to connect to the 5′ end of the aptamer, it significantly enhances the stability of the peptide linker vc in mouse plasma compared to connecting to both ends of the aptamer or to just the 3′ end.

### BRIEF DESCRIPTION OF THE DRAWINGS

FIG. 1 is the mass spectrometry characterization of the PTK7-targeted ApDC S4M with a drug-loading ratio of 4 from Example 1.

FIG. 2 is the mass spectrometry characterization of the PTK7-targeted ApDC S2M with a drug-loading ratio of 2 from Example 2.

FIG. 3 is the mass spectrometry characterization of the PTK7-targeted ApDC S3M with a drug-loading ratio of 3 from Example 3.

FIG. 4 is the mass spectrometry characterization of the CMET-targeted ApDC SL1-3M with a drug-loading ratio of 3 from Example 4.

FIG. 5 is the mass spectrometry characterization of the PTK7-targeted ApDC S2D with a drug-loading ratio of 2 from Example 5.

FIG. 6 is the mass spectrometry characterization of the CMET-targeted ApDC SL1-3D with a drug-loading ratio of 3 from Example 6.

FIG. 7 is the mass spectrometry characterization of the PTK7-targeted ApDC S4D with a drug-loading ratio of 4 from Example 7.

FIG. 8 is the mass spectrometry characterization of the PTK7-targeted ApDC S4E with a drug-loading ratio of 4 from Example 11.

FIG. 9 shows the proliferation inhibition experiment of different drug-loading ratios of the PTK7-targeted ApDC in ovarian cancer SKOV-3 cells from Example 12.

FIG. 10 presents the tumor volume curve in vivo for the inhibition experiment of different drug-loading ratios of the PTK7-targeted ApDC from Example 13.

FIG. 11 shows the mouse weight curve in vivo for the inhibition experiment of different drug-loading ratios of the PTK7-targeted ApDC from Example 13.

FIG. 12 presents the tumor volume curve in vivo for S4D from Example 14.

FIG. 13 shows the mouse weight curve in vivo for S4D from Example 14.

FIG. 14 displays images of mice in the in vivo tumor inhibition experiment using S4M from Example 15.

FIG. 15 illustrates the tumor volume curve in vivo for S4M from Example 15.

FIG. 16 presents the relative tumor volume curve in vivo for S4M from Example 15.

FIG. 17 shows the mouse weight curve in vivo for S4M from Example 15.

FIG. 18 presents the relative weight curve in vivo for S4M from Example 15.

FIG. 19 shows the tumor volume curve in vivo for S2M and S4M from Example 16.

FIG. 20 illustrates the relative tumor volume curve in vivo for S2M and S4M from Example 16.

FIG. 21 presents the tumor volume curve in vivo for S4M from Example 17.

FIG. 22 shows the mouse weight for S4M from Example 17.

FIG. 23 presents the tumor volume in the in vivo tumor inhibition experiment with different drug-loading ratios of the PTK7-targeted ApDC in the OVCAR3 model from Example 18.

FIG. 24 shows the mouse weight in the in vivo tumor inhibition experiment with different drug-loading ratios of the PTK7 ApDC in the OVCAR3 model from Example 18.

FIG. 25 illustrates the proliferation experiment of different drug-loading ratios of the PTK7 ApDC in NCI-H1975 cells from Example 19.

FIG. 26 presents the tumor volume curve in vivo for the different drug-loading ratios of the PTK7 ApDC in the HT-29 model from Example 20.

FIG. 27 shows mouse weight in the HT-29 model after in vivo tumor suppression experiments with PTK7-targeted ApDC with different drug-loading ratios in Example 20.

### DETAILED DESCRIPTION OF THE INVENTION

In conjunction with the attached figure and the implementation examples, a further detailed description of the present invention will be provided. It should be noted that the following described implementation examples aim to facilitate the understanding of the present invention and do not impose any limiting effect,

### Example 1: Preparation of the PTK7 ApDC S4M with a Drug-Loading Ratio of 4

This example describes the preparation of a PTK7 (sgc8c, disclosed sequence) ApDC S4M with a drug-loading ratio of 4, constructed by four conjugation sites of the PTK7 aptamer and the Maytansine analog microtubule inhibitor MMAE. The four conjugation sites of the PTK7 aptamer are prepared using solid-phase synthesis.

The ApDC S4M with a drug-loading ratio of 4 is synthesized using a thiol-maleimide chemical reaction, as shown in the reaction equation below (Where the thiol-containing strip is the aptamer):

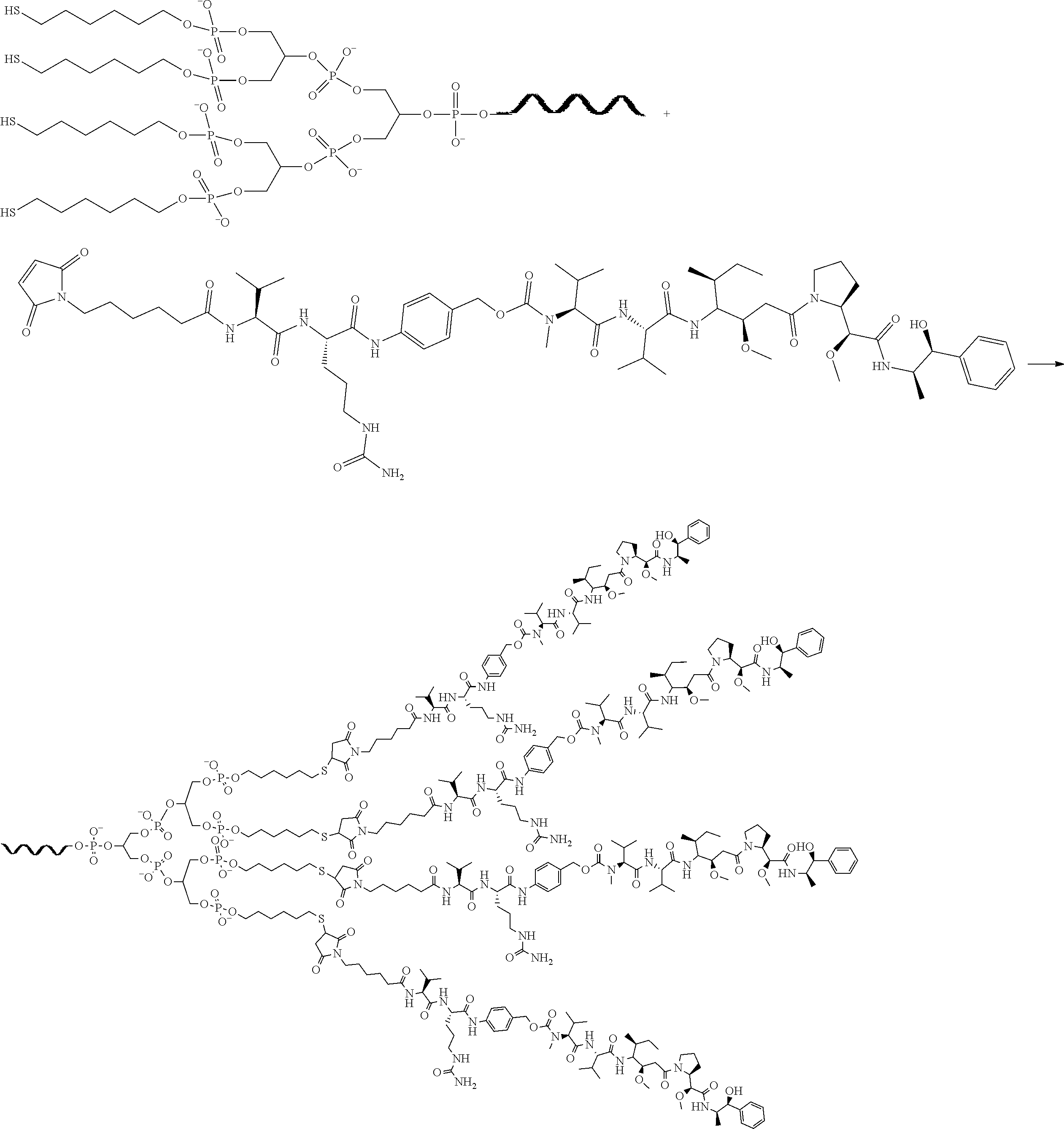

The preparation process is as follows:

(1) Preparation of the Aqueous Solution of the PTK7 Aptamer with Four Conjugation Sites:

First, the synthesis of the oligodeoxynucleotide chain (sgc8c) is performed using solid-phase synthesis technology. Controlled-pore glass (CPG) is selected as the solid-phase support for the synthesis reaction, which requires strict dehydration and deoxygenation of the synthesis reagents. The nucleic acid sequence (sgc8c: 5′-3′: ATC TAA CTG CTG CGC CGC CGG GAA AAT ACT GTA CGG TTA GA (SEQ NO: 1) is entered into the control software of the solid-phase synthesizer. At the 5′ end, the bifurcated phosphoramidite 1 (x=1) modifications were coupled twice to form 4 terminal hydroxyl groups for modification of four thiol group. The bifurcated phosphoramidite monomer is prepared at a concentration of 0.1 M for coupling. After twice coupling of the bifurcated linker, a thiol modification is performed, followed by treatment with 30% ammonium hydroxide to remove the protecting groups from the oligodeoxynucleotide chain and to hydrolyze it off the support. After heating, the reaction solution is cooled, and a 10% volume of 3 M sodium chloride solution is added based on the volume of ammonium hydroxide used. After mixing, anhydrous ethanol is added at 2.5 times the volume of ammonium hydroxide used. After mixing again, a white flocculent precipitate appears, which is the crude product obtained from the synthesis. The supernatant is removed by centrifugation, and the precipitate is dried as much as possible. Pure water is then added for re-dissolution, and after filtration, the crude product is obtained, which is purified using HPLC, collecting the corresponding fractions. At this point, the thiol groups of the aptamer product are protected by disulfide bonds, which are reduced using 20 equivalents of TCEP, followed by desalting to remove small molecules, resulting in an aqueous solution of the aptamer with four conjugation sites.

(2) Preparation of the ApDC S4M with a Drug-Loading Ratio of 4:

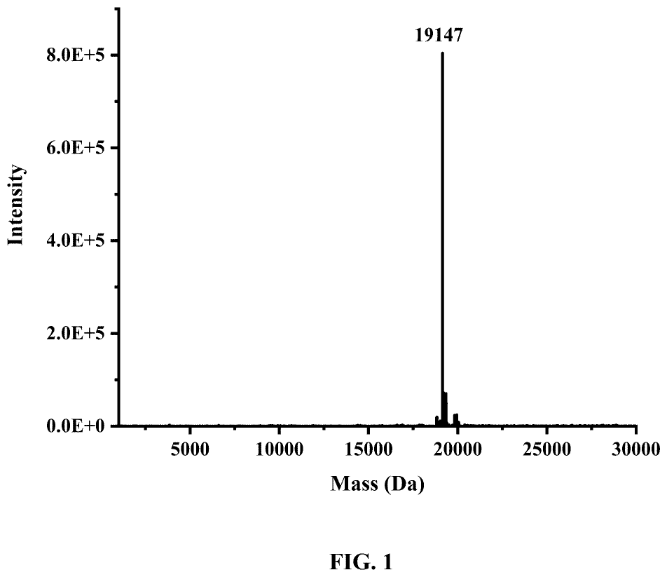

An aqueous solution of the PTK7 aptamer with four conjugation sites (1 equivalent) is added to a centrifuge tube along with a mixed solution of the Maytansine analog microtubule inhibitor MMAE in acetonitrile/water (1/5-3/1, preferably 1/1) at 2-30 equivalents (preferably 15 equivalents). The temperature is maintained at 4-40° C., preferably at 25° C., and the reaction is stirred for 2-24 hours, preferably for 12 hours. Reverse-phase preparative column purification is performed, and freeze-drying produces the PTK7 ApDC S4M (Sgc8c-4MMAE) with a drug-loading ratio of 4, yielding approximately 70%. After desalting, it is freeze-dried for storage. The mass spectrum is shown in FIG. 1, MS: Calculated: 19146.4 (Found: 19147.0).

### Example 2: Preparation of the PTK7 ApDC S2M with a Drug-Loading Ratio of 2

This example describes the preparation of a PTK7 ApDC S2M with a drug-loading ratio of 2, constructed from a PTK7 aptamer with two conjugation sites and the Maytansine analog microtubule inhibitor MMAE. The PTK7 aptamer with two conjugation sites is prepared using solid-phase synthesis.

The ApDC S2M with a drug-loading ratio of 2 is synthesized using a thiol-maleimide chemical reaction, as shown in the reaction equation below:

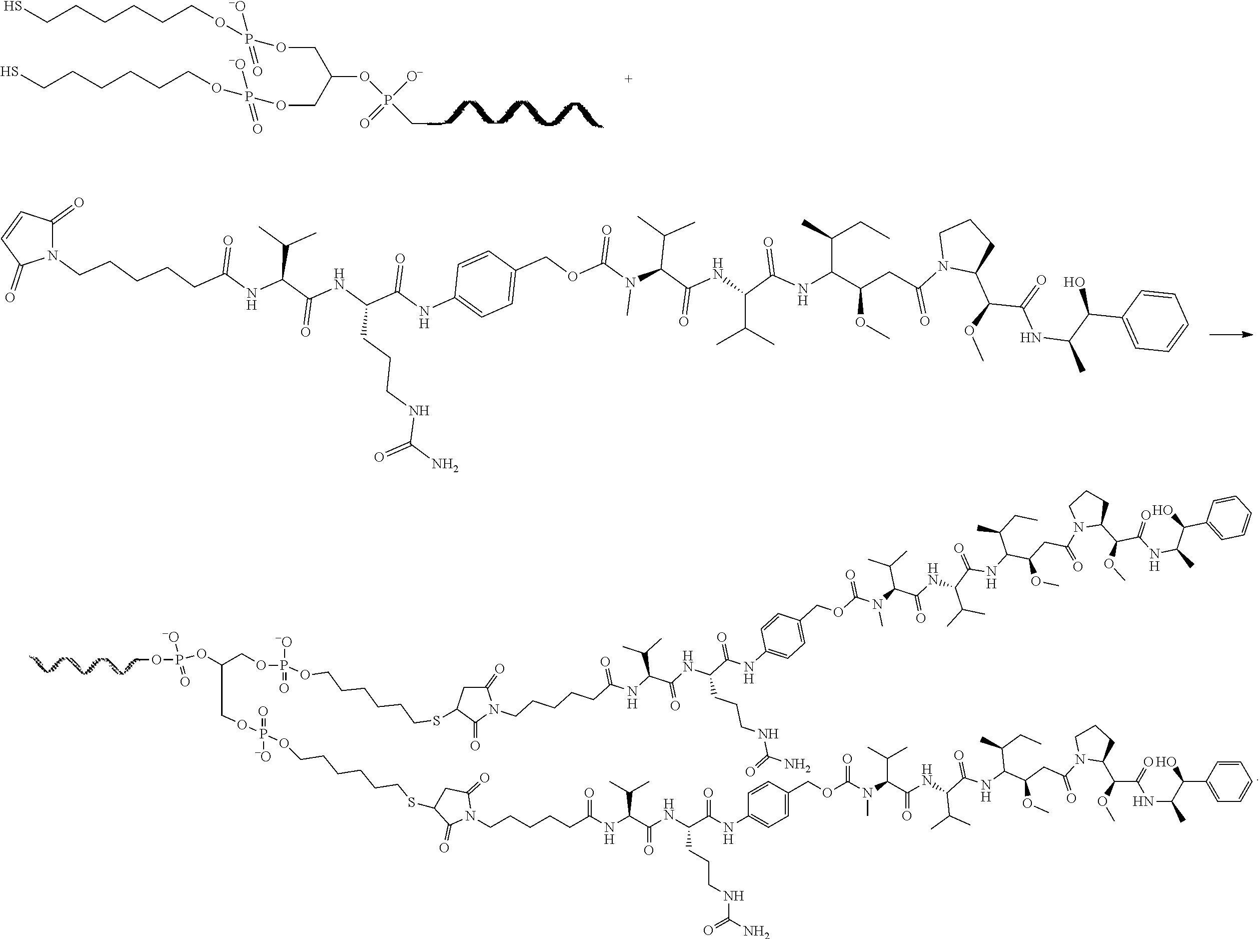

The preparation process is as follows:

(1) Preparation of the Aqueous Solution of the PTK7 Aptamer with Two Conjugation Sites:

The synthesis of the oligodeoxynucleotide chain was initially performed using solid-phase synthesis technology. Controlled-pore glass (CPG) was utilized as the solid-phase support for the synthesis reaction, necessitating rigorous dehydration and deoxygenation of the synthesis reagents. The nucleic acid sequence (sgc8c: 5′-3′: ATC TAA CTG CTG CGC CGC CGG GAA AAT ACT GTA CGG TTA GA) was input into the control software of the solid-phase synthesizer. At the 5′ end, bifurcated phosphoramidite 1 (x=1) modifications were coupled once to generate two terminal hydroxyl groups for the modification of two thiol groups. The bifurcated phosphoramidite monomer was prepared at a concentration of 0.1 M for coupling. Following the initial coupling of the bifurcated linker, a thiol modification was conducted, followed by treatment with 30% ammonium hydroxide to remove the protecting groups from the oligodeoxynucleotide chain and to hydrolyze it from the support. The subsequent processing steps were consistent with those outlined in Example 1. After purification and reduction, an aqueous solution of the aptamer containing two conjugation sites was obtained.

(2) Preparation of the ApDC S2M with a Drug-Loading Ratio of 2:

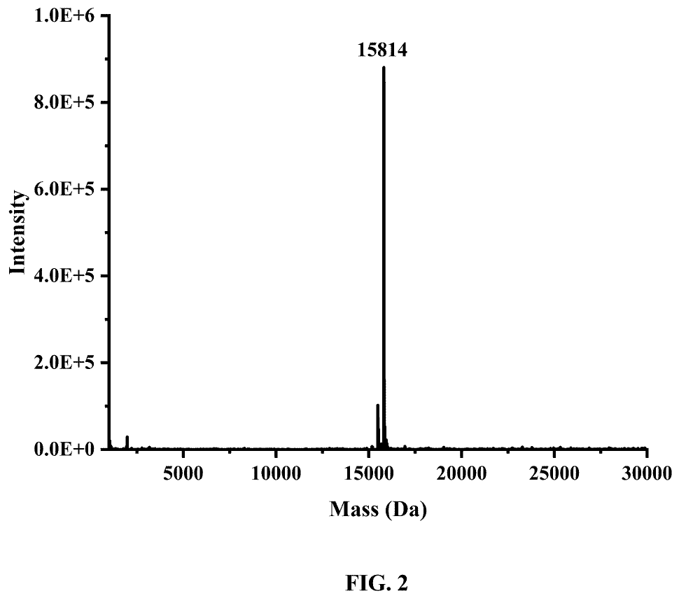

An aqueous solution of the PTK7 aptamer with two conjugation sites (1 equivalent) is added to a centrifuge tube along with a mixed solution of the Maytansine analog microtubule inhibitor MMAE in acetonitrile/water (1/5-3/1, preferably 1/1) at 2-20 equivalents (preferably 8 equivalents). The temperature is maintained at 4-40° C., preferably at 25° C., and the reaction is stirred for 2-24 hours, preferably for 16 hours. Reverse-phase preparative column purification is performed, and freeze-drying produces the PTK7 ApDC S2M (Sgc8c-2MMAE), yielding approximately 80%. After desalting, it is freeze-dried for storage. The mass spectrum is shown in FIG. 2, MS: Calculated: 15817.0 (Found: 15814.0).

### Example 3: Preparation of the PTK7 ApDC S3M with a Drug-Loading Ratio of 3

This example describes the preparation of a PTK7 ApDC S3M, constructed from a PTK7 aptamer with triple conjugation sites and the Maytansine analog microtubule inhibitor MMAE. The PTK7 aptamer with three conjugation sites is prepared using solid-phase synthesis.

The ApDC S3M with a drug-loading ratio of 3 is synthesized using a thiol-maleimide chemical reaction, as shown in the reaction equation below:

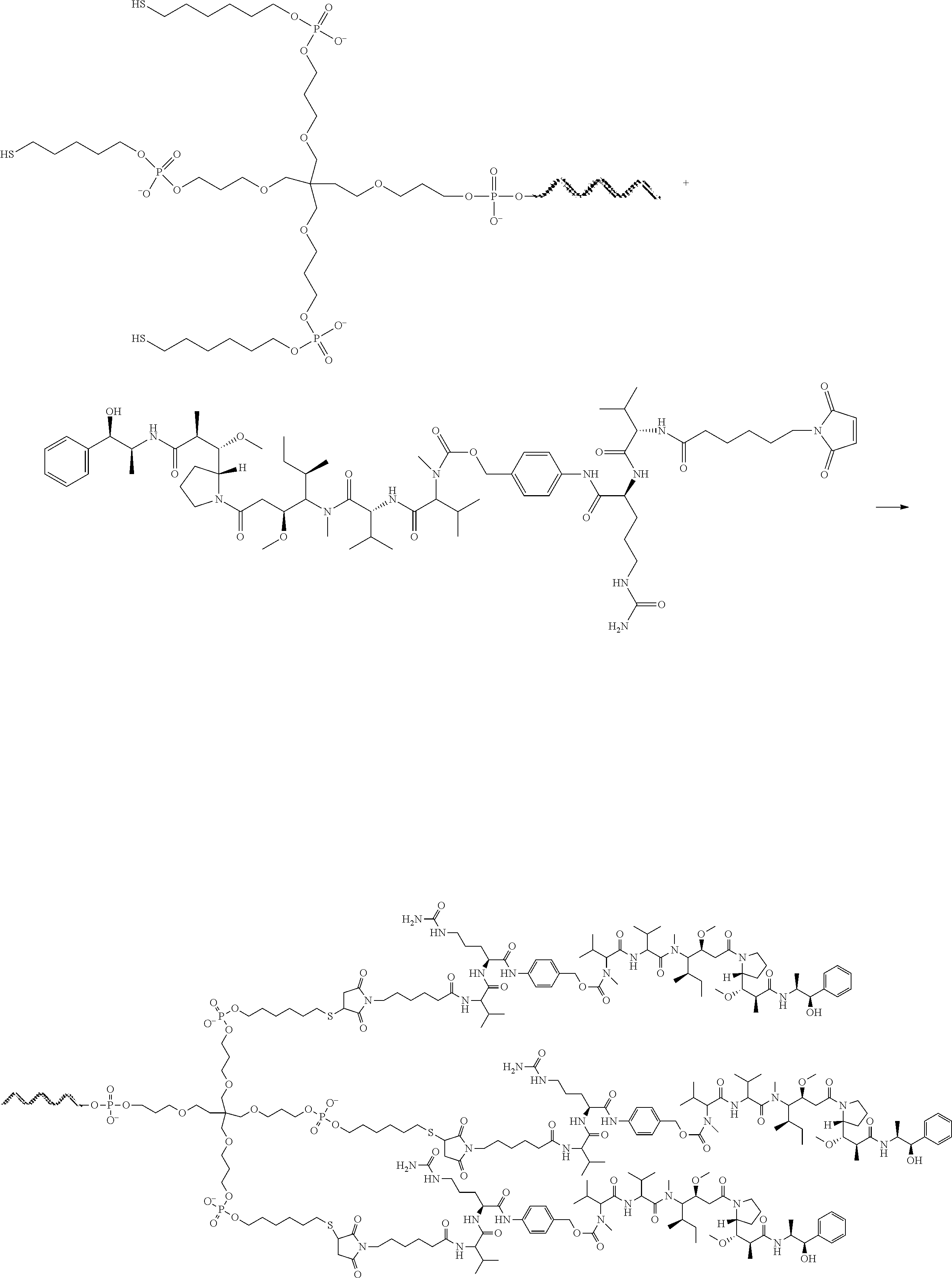

The preparation process is as follows:

(1) Preparation of the Aqueous Solution of the PTK7 Aptamer with Three Conjugation Sites:

The synthesis of the oligodeoxynucleotide chain was initially performed using solid-phase synthesis technology. Controlled-pore glass (CPG) was utilized as the solid-phase support for the synthesis reaction, necessitating rigorous dehydration and deoxygenation of the synthesis reagents. The nucleic acid sequence (sgc8c: 5′-3′: ATC TAA CTG CTG CGC CGC CGG GAA AAT ACT GTA CGG TTA GA) was input into the control software of the solid-phase synthesizer. At the 5′ end, a trifurcated phosphoramidite 3 modification (x=3) modification was coupled once to generate three terminal hydroxyl groups for the modification of three thiol groups. The trifurcated phosphoramidite monomer was prepared at a concentration of 0.1 M for coupling. Following the initial coupling of the trifurcated linker, a thiol modification was conducted, followed by treatment with 30% ammonium hydroxide to remove the protecting groups from the oligodeoxynucleotide chain and to hydrolyze it from the support. The subsequent processing steps were consistent with those outlined in Example 1. After purification and reduction, an aqueous solution of the aptamer containing three conjugation sites was obtained.

(2) Preparation of the ApDC S3M with a Drug-Loading Ratio of 3:

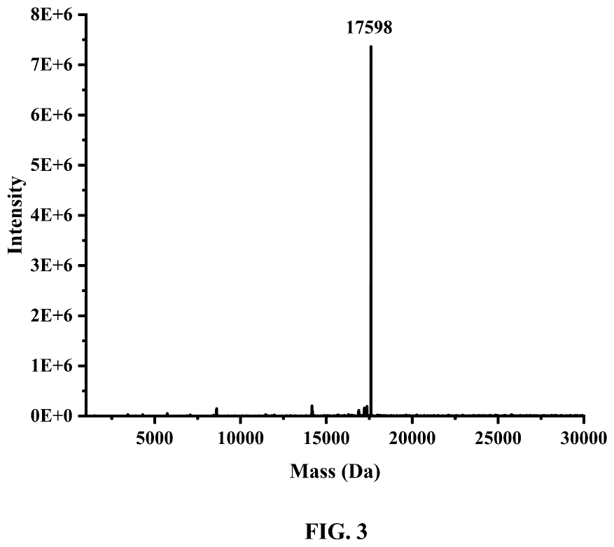

An aqueous solution of the PTK7 aptamer Sgc8c with three conjugation sites (1 equivalent) is added to a centrifuge tube along with a mixed solution of the Maytansine analog microtubule inhibitor MMAE in acetonitrile/water (1/5-3/1, preferably 1/1) at 2-20 equivalents (preferably 12 equivalents). The temperature is maintained at 4-40 C°, preferably at 25 C°, and the reaction is stirred for 2-24 hours, preferably 16 hours. Reverse-phase preparative column purification is performed, and freeze-drying produces the PTK7 ApDC S3M (Sgc8c-3MMAE) with a drug-loading ratio of 3, yielding approximately 75%. After desalting, it is freeze-dried for storage. The mass spectrum is shown in FIG. 3, MS: Calculated: 17599.0 (Found: 17598.0).

### Example 4: Preparation of the CMET ApDC SL1-3M with a Drug-Loading Ratio of 3

This example describes the preparation of a CMET ApDC SL1-3M with a drug-loading ratio of 3, constructed from a CMET aptamer with three conjugation sites and the Maytansine analog microtubule inhibitor MMAE. The CMET aptamer with three conjugation sites is prepared using solid-phase synthesis.

The ApDC SL1-3M with a drug-loading ratio of 3 is synthesized using a thiol-maleimide chemical reaction, as shown in the reaction equation below:

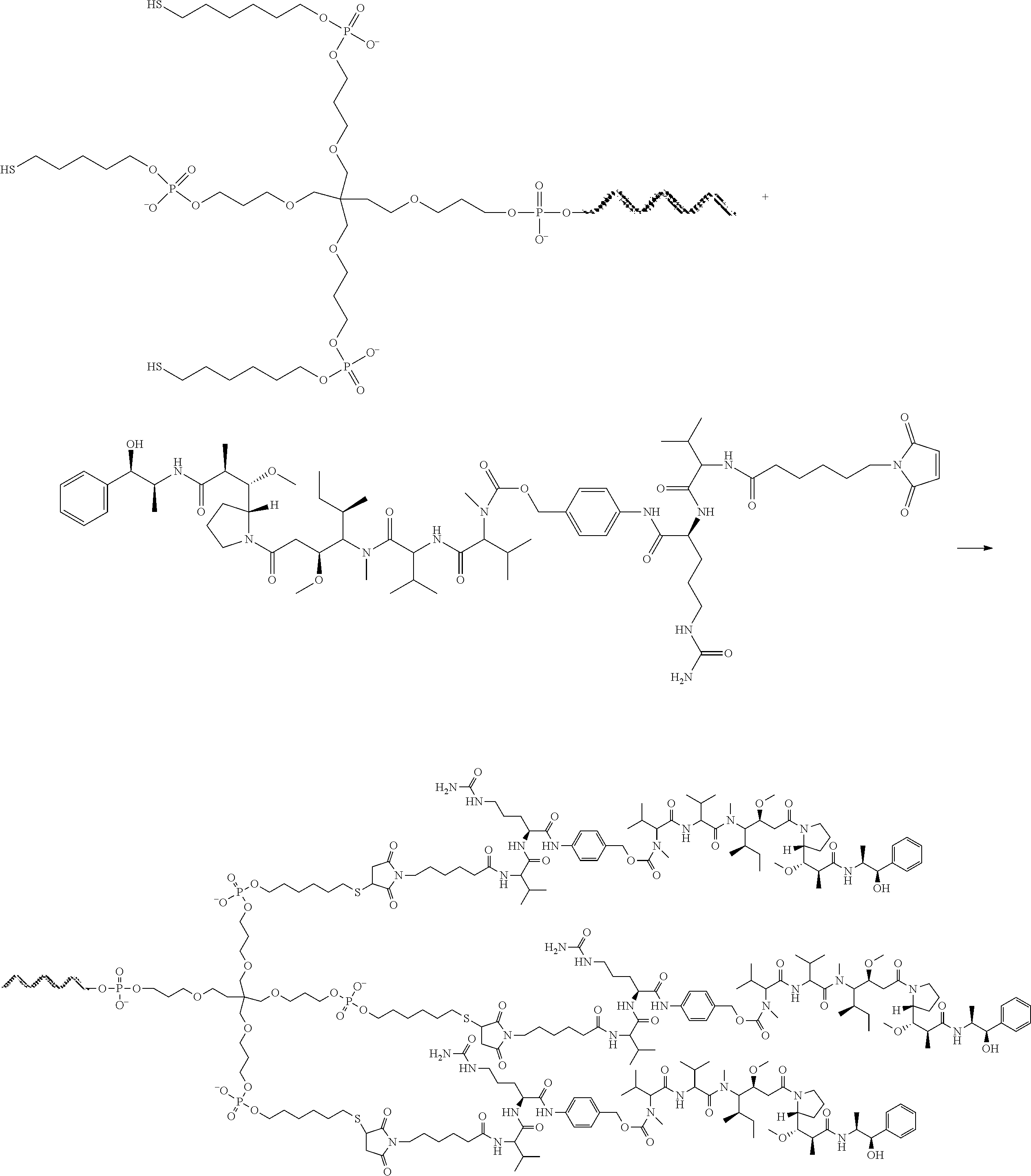

The preparation process closely follows the methodology described in Example 3, with the substitution of PTK7-targeted sgc8c by the CMET-targeted aptamer SL1 (SL-1: ATCAGGCTGGATGGTAGCTCGGTCGGGGTGGGTGGGTTGGCAAGTCTGAT) (SEQ NO: 2). The resulting CMET-targeted aptamer-drug conjugate (ApDC) SL1-3M (SL1-3MMAE) is synthesized with a drug-loading ratio of 3, achieving an approximate yield of 78%. Subsequent to desalting, the product is freeze-dried for storage. The mass spectrum analysis is presented in FIG. 4, with the calculated mass being 20646.0 and the observed mass being 20647.0.

### Example 5: Preparation of the PTK7 ApDC S2D with a Drug-Loading Ratio of 2

This example describes the preparation of a PTK7 ApDC S2D with a drug-loading ratio of 2, constructed from a PTK7 aptamer with two conjugation sites and the DNA topoisomerase I inhibitor Dxd. The PTK7 aptamer is prepared using solid-phase synthesis.

The ApDC S2D with a drug-loading ratio of 2 is synthesized using a thiol-maleimide chemical reaction, as shown in the reaction equation below:

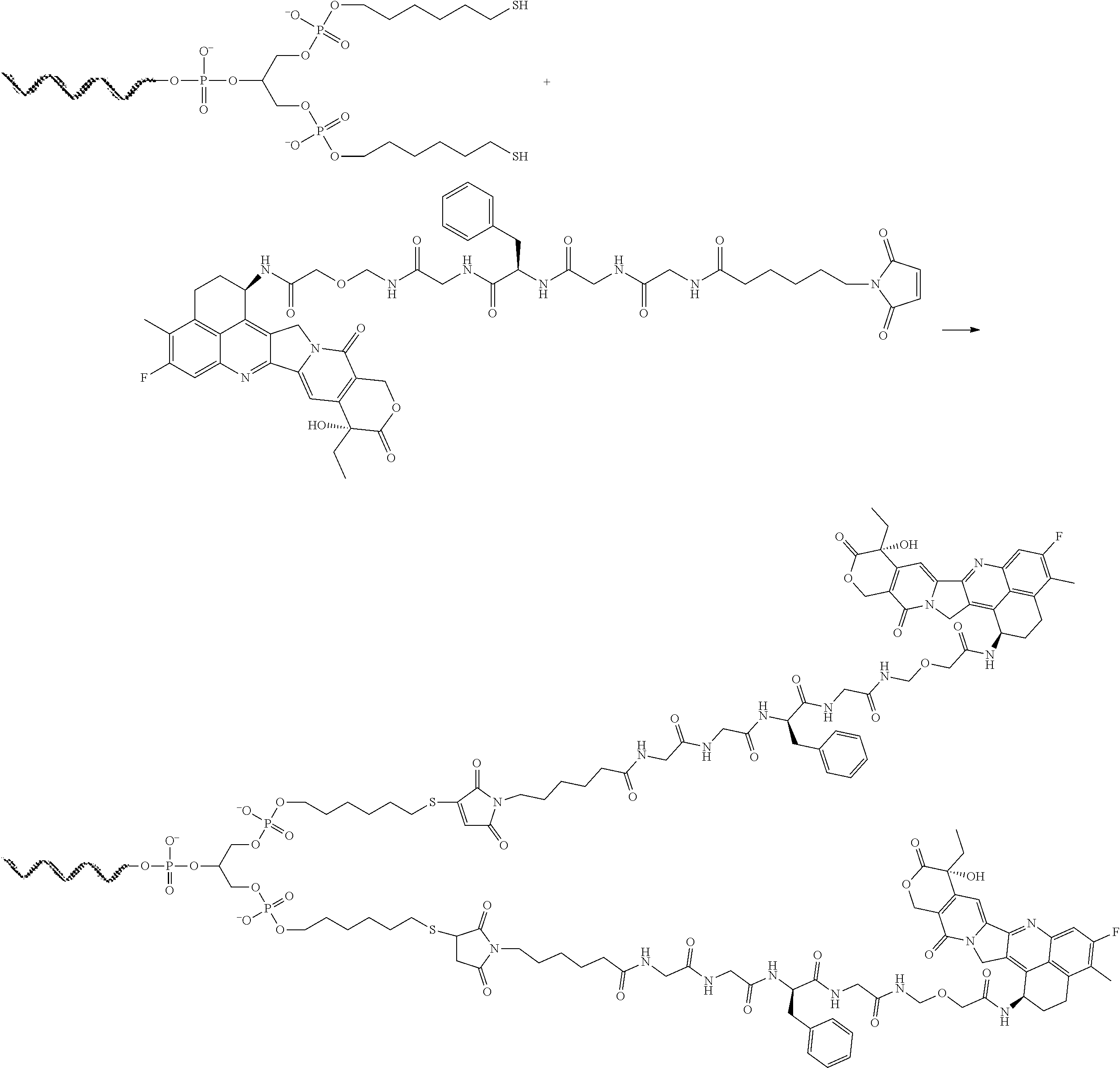

The preparation process is as follows:
(1) The preparation of the aqueous solution of the PTK7 aptamer with two conjugation sites is as described in Example 2, but the sequence of the aptamer is SEQ NO: 1.
(2) Preparation of the ApDC S2D with a drug-loading ratio of 2:

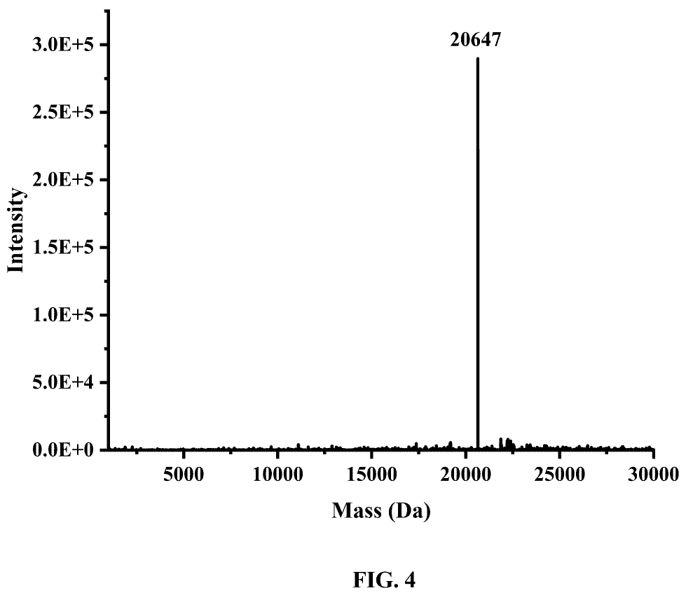

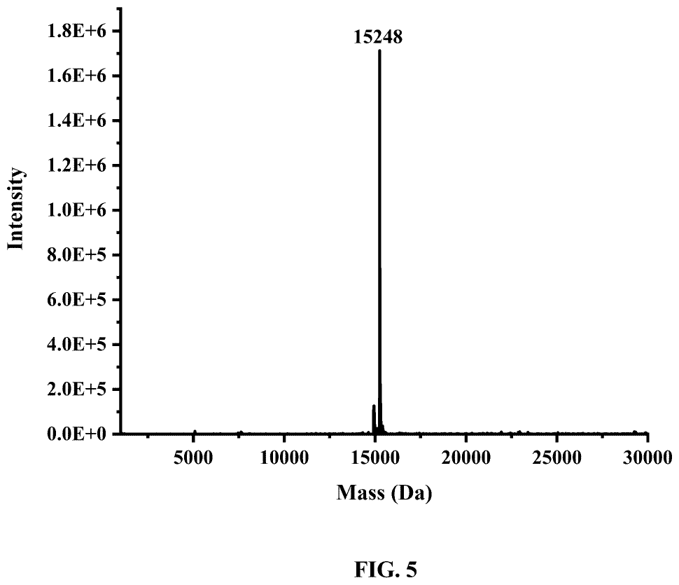

An aqueous solution of the PTK7 aptamer with two conjugation sites (1 equivalent) is added to a centrifuge tube along with a mixed solution of the DNA topoisomerase I inhibitor Dxd in acetonitrile/water (1/5-3/1, preferably 1/1) at 2-20 equivalents (preferably 8 equivalents). The temperature is maintained at 4-40 C°, preferably at 25 C°, and the reaction is stirred for 2-24 hours, preferably for 8 hours. Reverse-phase preparative column purification is performed, and freeze-drying produces the PTK7 ApDC S2D (Sgc8c-2Dxd) with a drug-loading ratio of 2, yielding approximately 70%. After desalting, it is freeze-dried for storage. The mass spectrum is shown in FIG. 5, MS: Calculated: 15253.0 (Found: 15248.0).

### Example 6: Preparation of the CMET ApDC SL1-3D with a Drug-Loading Ratio of 3

This example describes the preparation of a CMET ApDC SL1-3D with a drug-loading ratio of 3, constructed from a CMET aptamer with three conjugation sites and the DNA topoisomerase I inhibitor Dxd. The CMET aptamer is prepared using solid-phase synthesis.

The ApDC SL1-3D with a drug-loading ratio of 3 is synthesized using a thiol-maleimide chemical reaction, as shown in the reaction equation below:

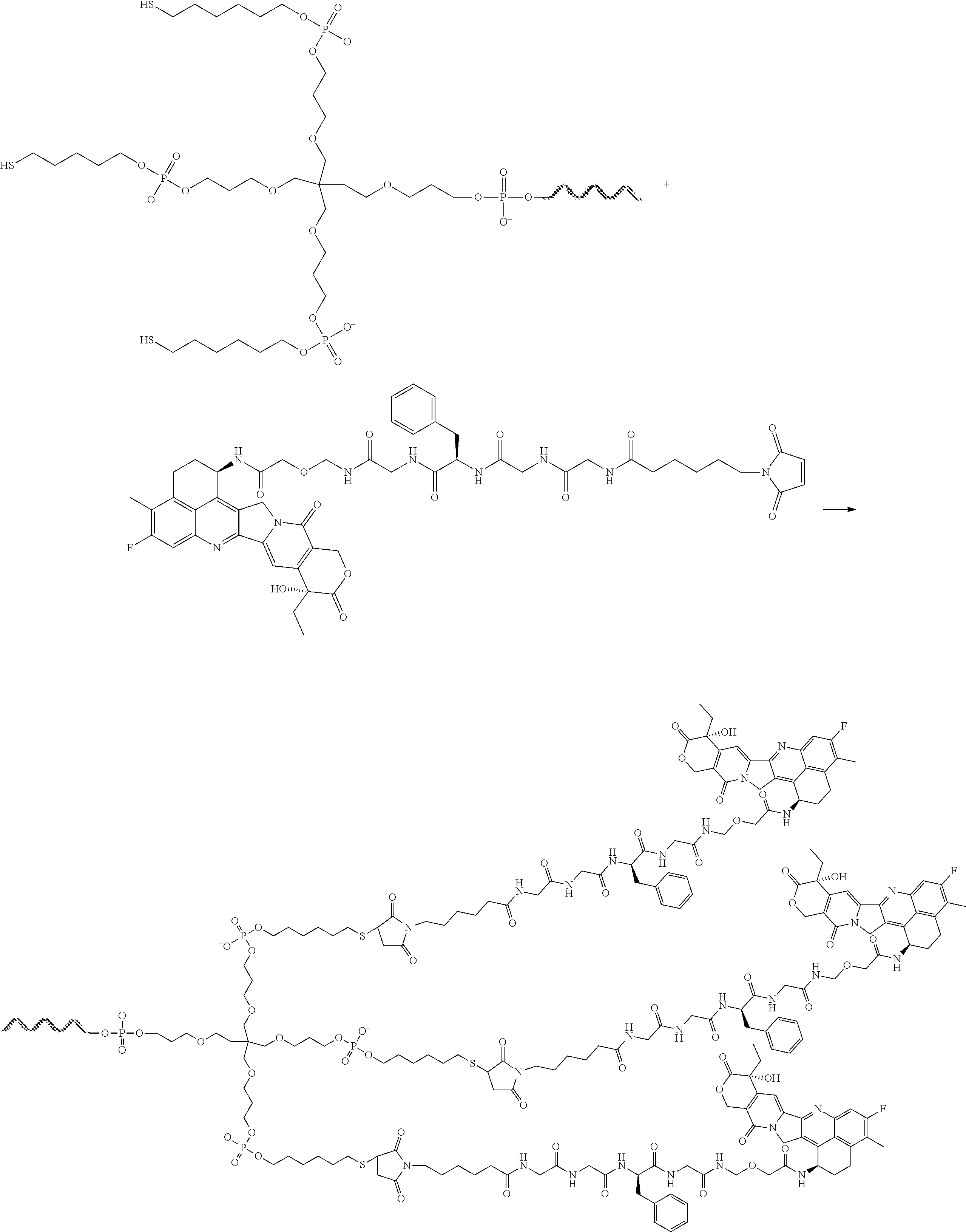

The preparation process is as follows:
(1) The preparation of the aqueous solution of the CMET aptamer with three conjugation sites is as described in Example 4, but the sequence of the aptamer is SEQ NO: 2.
(2) Preparation of the ApDC SL1-3D with a drug-loading ratio of 3:

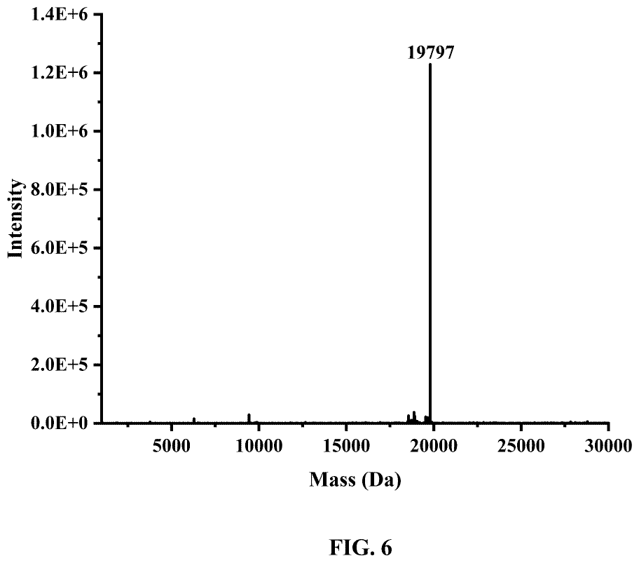

An aqueous solution of the CMET aptamer with three conjugation sites (1 equivalent) is added to a centrifuge tube along with a mixed solution of the DNA topoisomerase I inhibitor Dxd in acetonitrile/water (1/5-3/1, preferably 1/1) at 2-20 equivalents (preferably 12 equivalents). The temperature is maintained at 4-40 C°, preferably at 25 C°, and the reaction is stirred for 2-24 hours, preferably for 8 hours. Reverse-phase preparative column purification is performed, and freeze-drying produces the CMET ApDC SL1-3D (SL1-3Dxd) with a drug-loading ratio of 3, yielding approximately 70%. After desalting, it is freeze-dried for storage. The mass spectrum is shown in FIG. 6, MS: Calculated: 19800.0 (Found: 19797.0).

### Example 7: Synthesis the PTK7 ApDC S4D with a Drug-Loading Ratio of 4

This example describes the preparation of the PTK7 ApDC S4D with a drug-loading ratio of 4, constructed from a PTK7 aptamer with four conjugation sites and the DNA topoisomerase I inhibitor Dxd. The PTK7 aptamer is prepared using solid-phase synthesis. The ApDC S4D with a drug-loading ratio of 4 is synthesized using a thiol-maleimide chemical reaction, as shown in the reaction equation below:

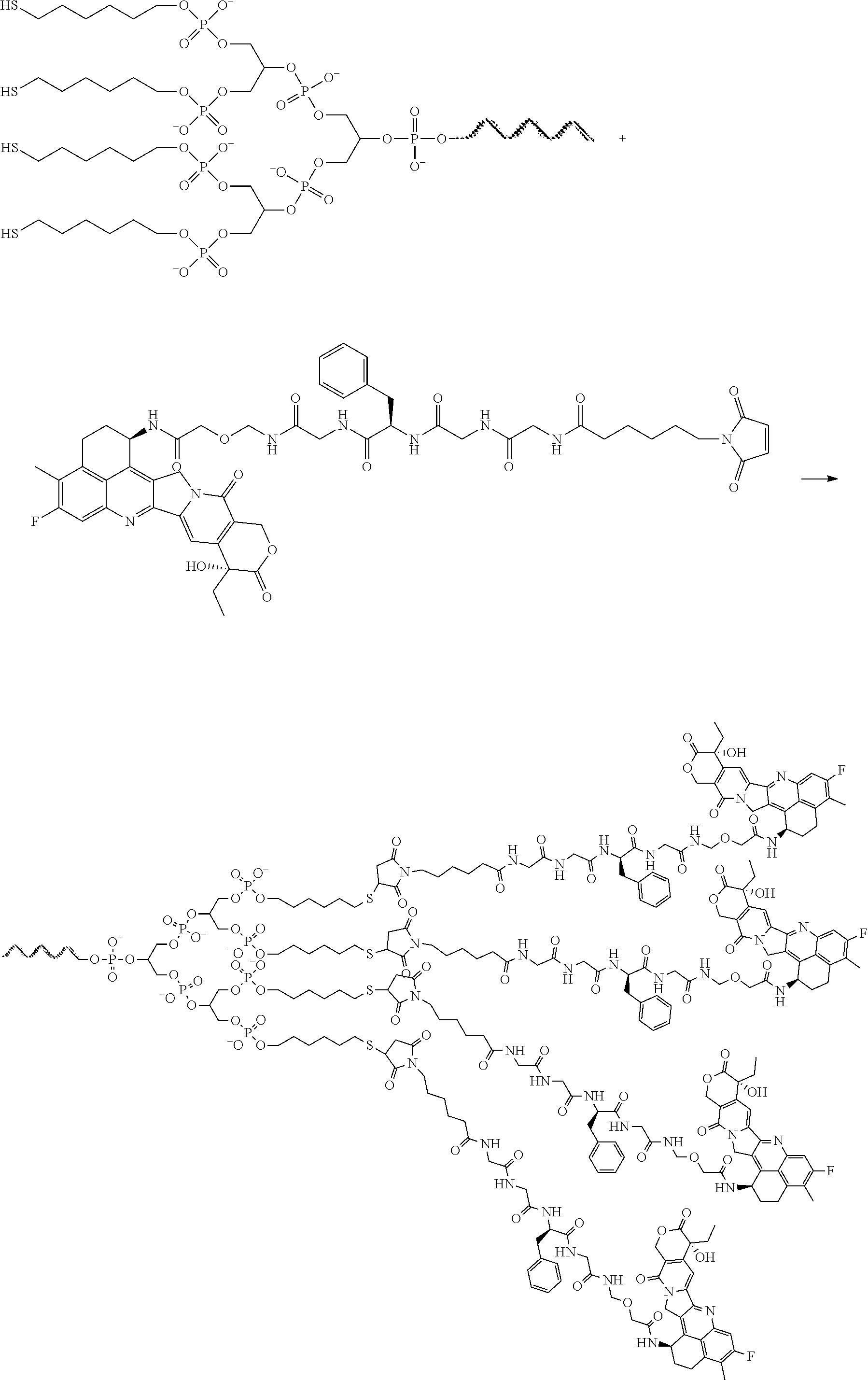

The preparation process is as follows:
(1) The preparation of the aqueous solution of the PTK7 aptamer with four conjugation sites is as described in Example 1, but the sequence of the aptamer is SEQ NO: 1.
(2) Preparation of the ApDC S4D with a drug-loading ratio of 4:

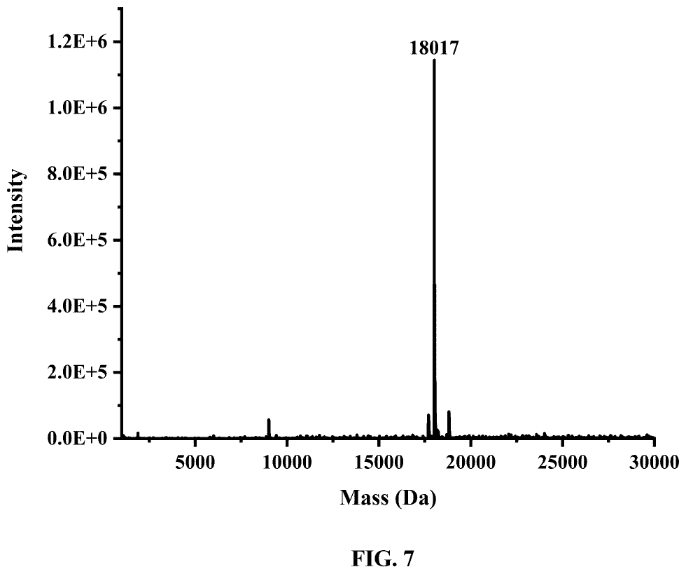

An aqueous solution of the PTK7 aptamer with four conjugation sites (1 equivalent) is added to a centrifuge tube along with a mixed solution of the DNA topoisomerase I inhibitor Dxd in acetonitrile/water (1/5-3/1, preferably 1/1) at 2-30 equivalents (preferably 15 equivalents). The temperature is maintained at 4-40 C°, preferably at 25 C°, and the reaction is stirred for 2-24 hours, preferably for 8 hours. Reverse-phase preparative column purification is performed, and freeze-drying produces the PTK7 ApDC S4D (Sgc8c-4Dxd) with a drug-loading ratio of 4, yielding approximately 70%. After desalting, it is freeze-dried for storage. The mass spectrum is shown in FIG. 7, MS: Calculated: 18016 (Found: 18017.0).

### Example 8: Synthesis of the PTK7 ApDC S3D with a Drug-Loading Ratio of 3

This example describes the preparation of the PTK7 ApDC S3D with a drug-loading ratio of 3, constructed from a PTK7 with three conjugation sites aptamer and the DNA topoisomerase I inhibitor Dxd. The PTK7 aptamer is prepared using solid-phase synthesis.

The ApDC S3D with a drug-loading ratio of 3 is synthesized using a thiol-maleimide chemical reaction, as shown in the reaction equation below:”

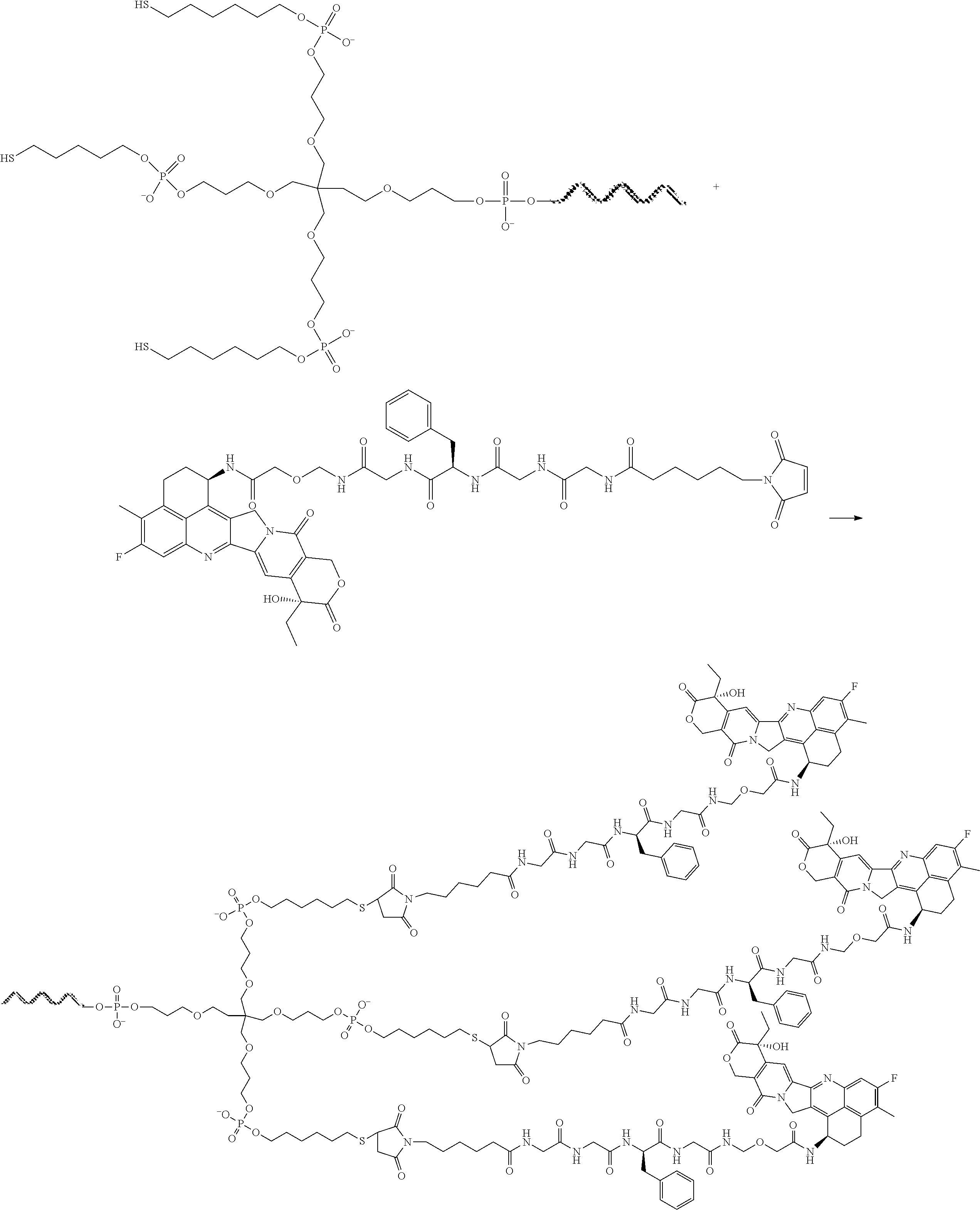

The preparation process is as follows:
(1) The preparation of the aqueous solution of the PTK7 aptamer with three conjugation sites is as described in Example 3, but the sequence of the aptamer is SEQ NO: 1.
(2) Preparation of the ApDC S3D with a drug-loading ratio of 3:

An aqueous solution of the PTK7 aptamer with three conjugation sites (1 equivalent) is added to a centrifuge tube along with a mixed solution of the DNA topoisomerase I inhibitor Dxd in acetonitrile/water (1/5-3/1, preferably 1/1) at 2-30 equivalents (preferably 15 equivalents). The temperature is maintained at 4-40 C°, preferably at 25 C°, and the reaction is stirred for 2-24 hours, preferably for 8 hours. Reverse-phase preparative column purification is performed, and freeze-drying produces the PTK7 ApDC S3D (Sgc8c-3Dxd) with a drug-loading ratio of 3, yielding approximately 70%. After desalting, it is freeze-dried for storage.

### Example 9: Synthesis of the PTK7 ApDC S6D with a Drug-Loading Ratio of 6

This example describes the preparation of the PTK7 ApDC S6D with a drug-loading ratio of 6, constructed from a PTK7 aptamer with six conjugation sites and the DNA topoisomerase I inhibitor Dxd. The PTK7 aptamer is prepared using solid-phase synthesis.

The ApDC S6D with a drug-loading ratio of 6 is synthesized using a thiol-maleimide chemical reaction, as shown in the reaction equation below:

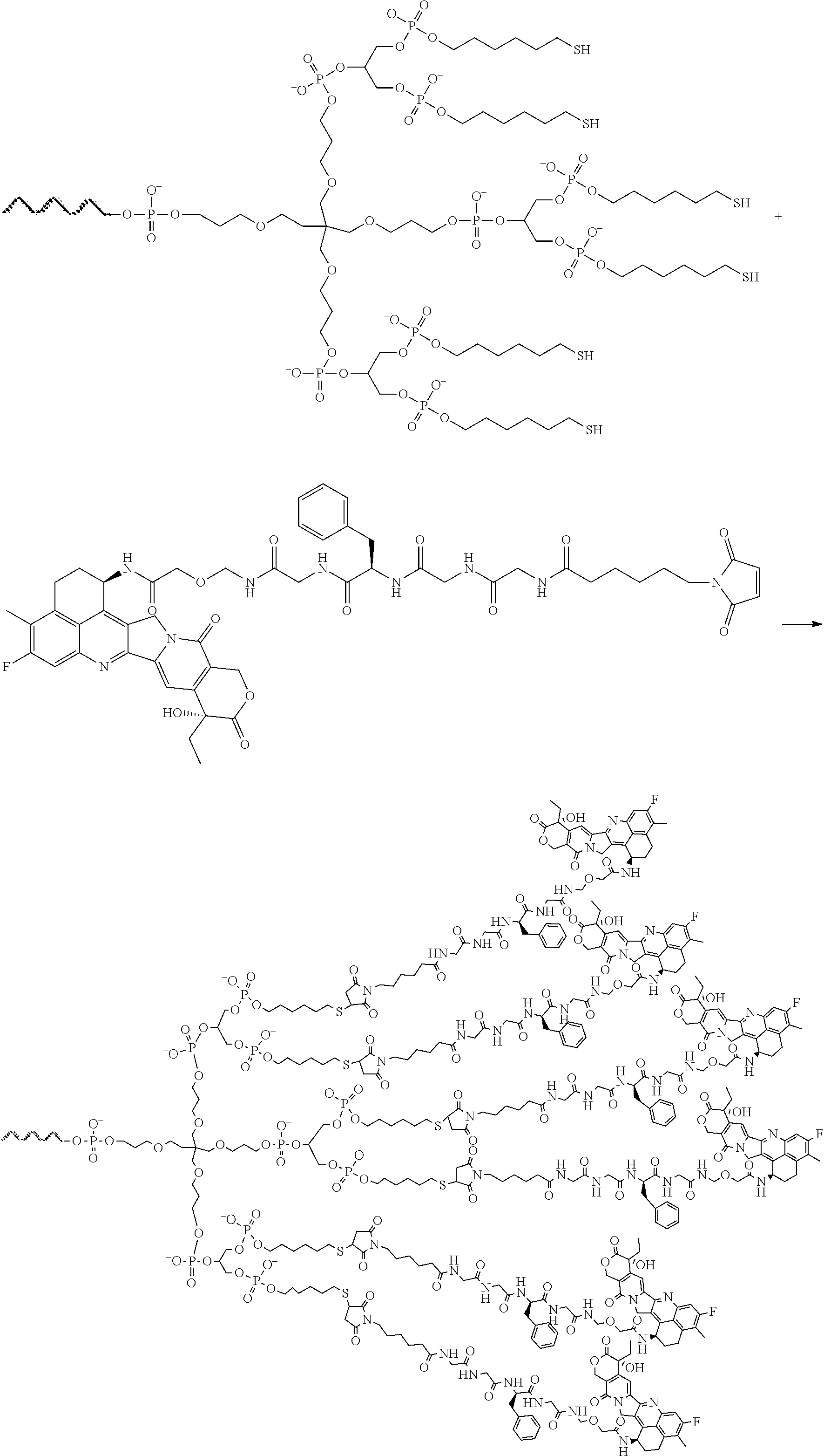

The preparation process is as follows:

(1) Preparation of the Aqueous Solution of the PTK7 Aptamer with Six Conjugation Sites:

The synthesis of the oligodeoxynucleotide chain (sgc8c-SEQ NO: 1) is conducted in accordance with the procedures outlined in Example 1. At the 5′ end, a trifurcated modification with phosphoramidite 3 (x=3) is introduced initially, followed by a bifurcated modification utilizing phosphoramidite 1 (x=1), then with thiol modifications. The phosphoramidite monomer is prepared at a concentration of 0.1 M for the purpose of coupling. After purification and reduction, an aqueous solution of the aptamer containing two conjugation sites was obtained.

(2) Preparation of the ApDC S6D with a Drug-Loading Ratio of 6:

An aqueous solution of the PTK7 aptamer with six conjugation sites (1 equivalent) is added to a centrifuge tube along with a mixed solution of the DNA topoisomerase I inhibitor Dxd in acetonitrile/water (1/5-3/1, preferably 1/1) at 2-30 equivalents (preferably 15 equivalents). The temperature is maintained at 4-40 C°, preferably at 25 C°, and the reaction is stirred for 2-24 hours, preferably for 8 hours. Reverse-phase preparative column purification is performed, and freeze-drying produces the PTK7 ApDC S6D (Sgc8c-6Dxd) with a drug-loading ratio of 6, yielding approximately 72%. After desalting, it is freeze-dried for storage.

### Example 10: Synthesis of the PTK7 ApDC S8D with a drug-loading ratio of 8

This example describes the preparation of the PTK7 ApDC S8D with a drug-loading ratio of 8, constructed from a PTK7 aptamer with eight conjugation sites and the DNA topoisomerase I inhibitor Dxd. The PTK7 aptamer is prepared using solid-phase synthesis.

The ApDC S8D with a drug-loading ratio of 8 is synthesized using a thiol-maleimide chemical reaction, as shown in the reaction equation below:

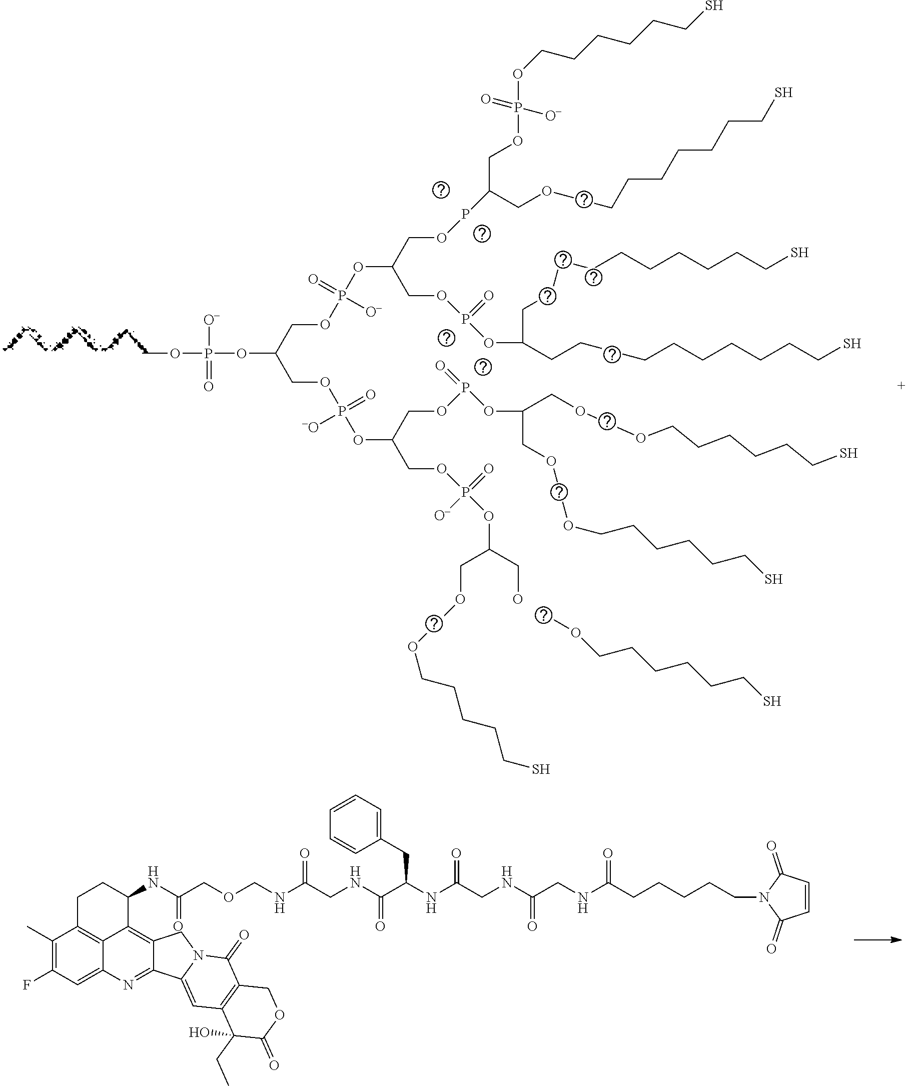

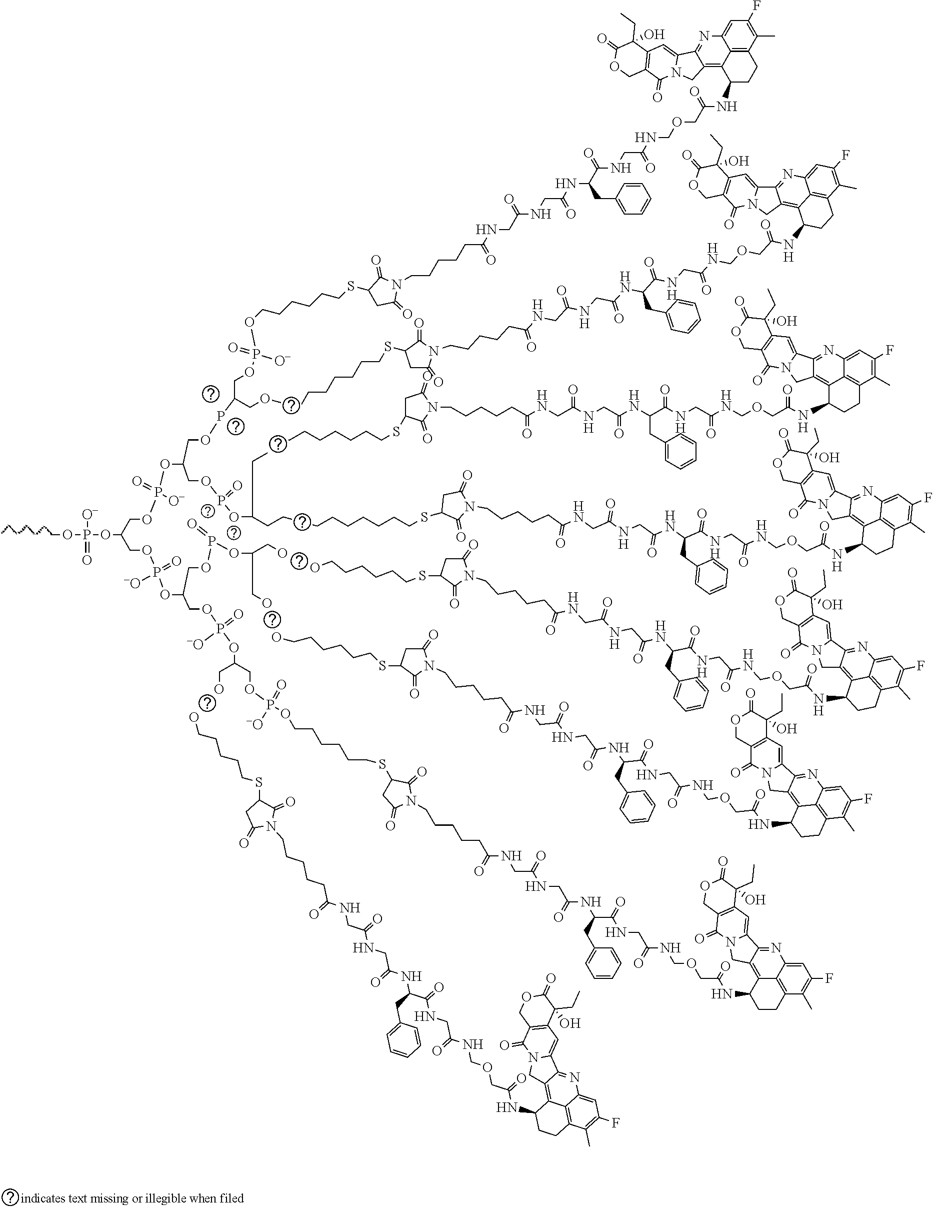

The preparation process is as follows:

(1) Preparation of the Aqueous Solution of the PTK7 Aptamer with Eight Conjugation Sites:

The synthesis of the oligodeoxynucleotide chain (sgc8c-SEQ NO: 1) is conducted in accordance with the procedures outlined in Example 1. At the 5′ end, a bifurcated modification phosphoramidite 1 (x=1) utilizing three times, followed with thiol modifications. The phosphoramidite monomer is prepared at a concentration of 0.1 M for the purpose of coupling. After purification and reduction, an aqueous solution of the aptamer containing two conjugation sites was obtained.

(2) Preparation of the ApDC S8D with a Drug-Loading Ratio of 8:

An aqueous solution of the PTK7 aptamer with eight conjugation sites (1 equivalent) is added to a centrifuge tube along with a mixed solution of the DNA topoisomerase I inhibitor Dxd in acetonitrile/water (1/5-3/1, preferably 1/1) at 2-30 equivalents (preferably 20 equivalents). The temperature is maintained at 4-40 C°, preferably at 25 C°, and the reaction is stirred for 2-24 hours, preferably for 8 hours. Reverse-phase preparative column purification is performed, and freeze-drying produces the PTK7 ApDC S8D (Sgc8c-8Dxd) with a drug-loading ratio of 8, yielding approximately 68%. After desalting, it is freeze-dried for storage.

### Example 11: Synthesis of the PTK7 ApDC S4E with a Drug-Loading Ratio of 4

This example describes the preparation of the PTK7 ApDC S4E with a drug-loading ratio of 4, constructed from a PTK7 aptamer with four conjugation sites and the DNA topoisomerase I inhibitor Exatecan. The PTK7 aptamer is prepared using solid-phase synthesis.

The ApDC S4E with a drug-loading ratio of 4 is synthesized using a thiol-maleimide chemical reaction, as shown in the reaction equation below:

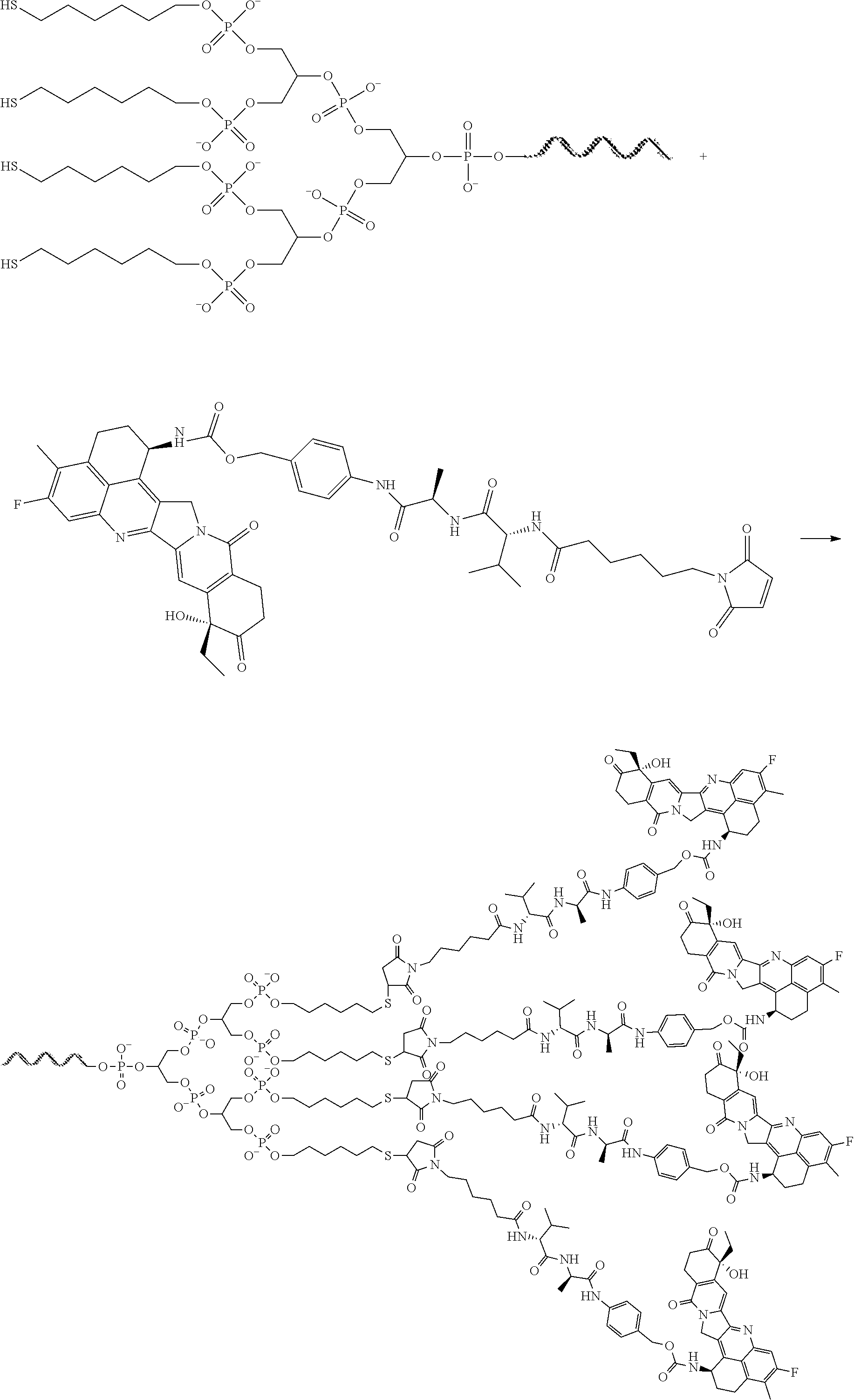

The preparation process is as follows:
(1) The preparation of the aqueous solution of the tetravalent PTK7 aptamer is as described in Example 1.
   (2) Preparation of the ApDC S4E with a Drug-Loading Ratio of 4:

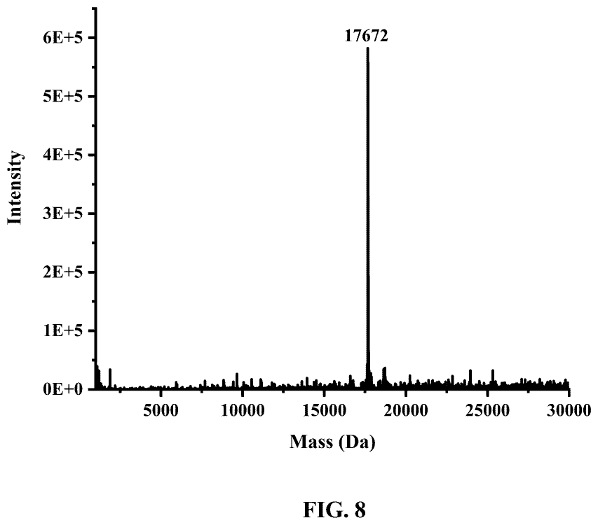

An aqueous solution of the PTK7 aptamer with four conjugation sites (1 equivalent) is added to a centrifuge tube along with a mixed solution of the DNA topoisomerase I inhibitor Exatecan in acetonitrile/water (1/5-3/1, preferably 2/1) at 2-30 equivalents (preferably 15 equivalents). The temperature is maintained at 4-40 C°, preferably at 25 C°, and the reaction is stirred for 2-24 hours, preferably for 12 hours. Reverse-phase preparative column purification is performed, and freeze-drying produces the PTK7 ApDC S4E (Sgc8c-4EXA) with a drug-loading ratio of 4, yielding approximately 65%. After desalting, it is freeze-dried for storage. The mass spectrum is shown in FIG. 8, MS: Calculated: 17672.0 (Found: 17672.0).

This example also further prepares a bifunctional PTK7 aptamer using the method provided in Example 2, resulting in the ApDC S2E with a drug-loading ratio of 2, which was validated by mass spectrometry.”

### Example 12: The Effect of Different Drug-Loading Ratios of ApDCs on the Proliferation Inhibition of Ovarian Cancer SKOV-3 Cells

In this example, S2D and S4D prepared in Examples 5 and 7, as well as the SD prepared by directly conjugating the PTK7 aptamer with the DNA topoisomerase I inhibitor Dxd (obtained by reacting the thiol-modified sgc8c aqueous solution from solid-phase synthesis with maleimide-modified Deruxtecan), were used for the proliferation inhibition experiment on ovarian cancer SKOV-3 cells. The specific process is as follows:

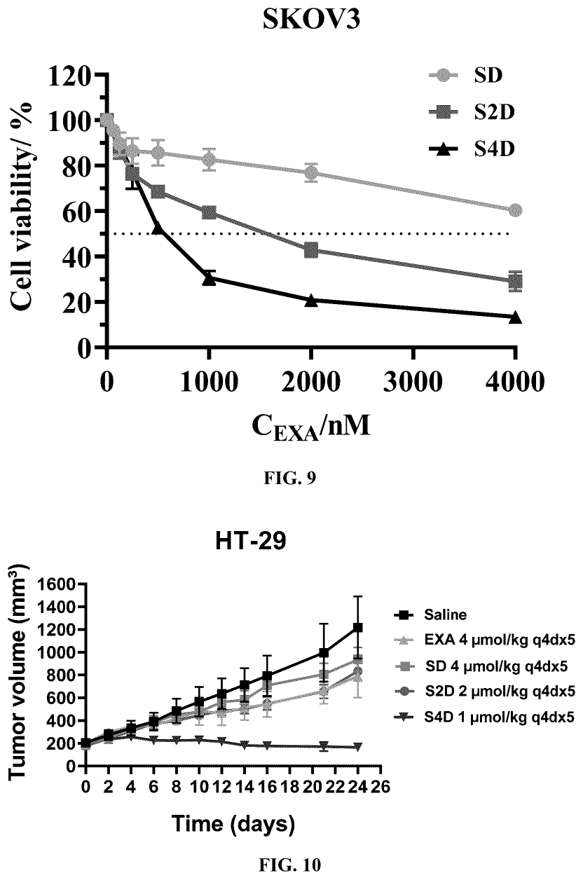

SKOV-3 cells were inoculated into a 96-well plate (4000 cells per well) and cultured for 24 hours at 37° C. in a 5% CO₂ environment. Different drug-loading ratio PTK7 ApDCs SD (Sgc8c-Dxd), S2D (Sgc8c-2Dxd), and S4D (Sgc8c-4Dxd) containing the same drug concentration were added. After 24 hours of cultivation at 37° C. with 5% CO₂, the drug-containing medium was discarded, and a drug-free medium was used to continue culturing for another 108 hours. After a total of 120 hours, the drug's inhibitory effect was measured using the MTS reagent kit, and the results are shown in FIG. 9. As shown in FIG. 9, the inhibition effects of different drug-loading ratio ApDCs SD, S2D, and S4D gradually increased with higher drug loading against ovarian cancer SKOV-3 cells, with the order of inhibitory activity being S4D>S2D>SD. The IC50 of S4D was reduced by over 8 times compared to SD.

### Example 13: The Effect of Different Drug-Loading Ratios of ApDCs on the Tumor Suppression of Colon Cancer HT-29

In this example, S2D and S4D prepared in Examples 5 and 7, as well as the SD prepared by directly conjugating the PTK7 aptamer with the DNA topoisomerase I inhibitor Dxd (obtained by reacting the thiol-modified sgc8c aqueous solution from solid-phase synthesis with maleimide-modified Deruxtecan), were used for tumor suppression experiments on the colon cancer HT-29 xenograft model. The specific process is as follows:

Mice bearing human colon cancer HT-29 tumors, with tumor volumes between 100-200 mm³, were randomly divided into eight groups. Treatments were administered every four days, with mice receiving either saline, the clinical frontline chemotherapy drug Exatecan (4 μmol/kg), the PTK7-targeted ApDC SD with a drug-loading ratio of 1 (dose 4 μmol/kg equivalent Dxd), S2D with a drug-loading ratio of 2 (dose 4 μmol/kg equivalent Dxd), S3D with a drug-loading ratio of 3 (dose 4 μmol/kg equivalent Dxd), S4D with a drug-loading ratio of 4 (dose 4 μmol/kg equivalent Dxd), S6D with a drug-loading ratio of 6 (dose 4 μmol/kg equivalent Dxd), and S8D with a drug-loading ratio of 8 (dose 4 μmol/kg equivalent Dxd), for a total of five injections. Mouse body weights, tumor lengths (a), and tumor widths (b) were measured and recorded at each injection. The tumor volume (V) was calculated using the formula: V=(a×b²)/2. The experiment was terminated when tumor volume exceeded 1500 mm³ or body weight reduced by more than 15%, at which point euthanasia was performed.

The results are shown in FIG. 10, indicating that different drug-loading ratio ApDCs exhibit varying antitumor efficacy. At the same drug dose, the antitumor efficacy gradually increases with the drug-loading ratio. This example also further verified the effects of S3D, S6D, and S8D (produced in Examples 8, 9, and 10) and found that beyond a drug-loading ratio of 4, there were no significant changes in antitumor effects, but systemic toxicity significantly increased. This may be due to the structure of the S4D drug being the most suitable for exerting its tumor-inhibiting effects at this drug dosage, with relatively low side effects, while other structurally different ApDCs were unable to achieve this effect. The S4D drug demonstrated the best in vivo tumor inhibition effect, allowing the DNA topoisomerase I inhibitor Dxd to achieve, and even exceed, the in vivo tumor inhibition effect of Exatecan, achieving over 95% tumor suppression efficiency.

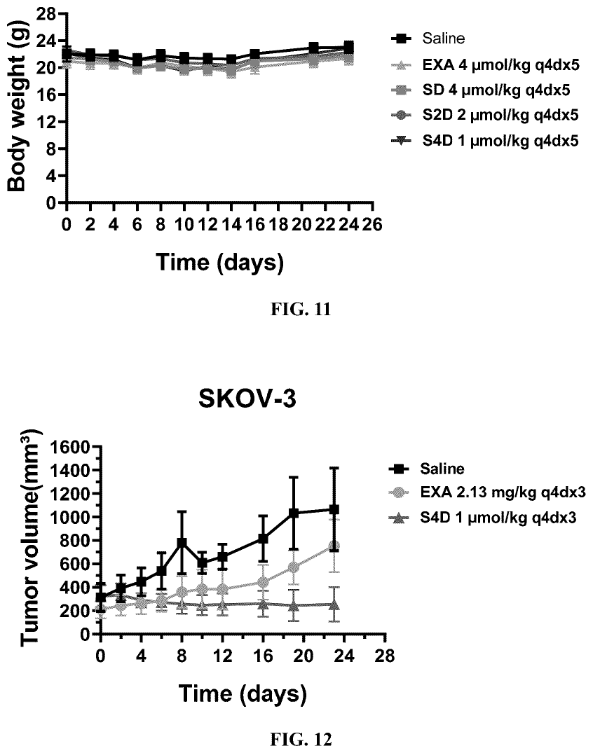

Simultaneously, mouse body weight was monitored, and the results are shown in FIG. 11. There was no significant difference in body weight between the S4D group and the saline group at this dosage, indicating that the high drug-loading ratio ApDCs have good biological safety at this dosage.

### Example 14: The Effect of S4D Concentration on Tumor Suppression of Ovarian Cancer SKOV-3 Cells

Twelve mice with subcutaneous tumor volumes of 200-400 mm³ bearing ovarian cancer SKOV-3 cells were randomly divided into four groups. Treatments were administered every four days, with mice receiving either saline, Exatecan, or the ApDC S4D (Sgc8c-4Dxd) at a drug-loading ratio of 4 (1 μmol/kg equivalent Dxd), for a total of three injections. Mouse body weights, tumor lengths (a), and tumor widths (b) were measured and recorded for each two days. The tumor volume (V) was calculated using the formula: V=(a×b²)/2. The experiment was terminated when tumor volume exceeded 1500 mm³ or body weight decreased by more than 15%, at which point euthanasia was performed.

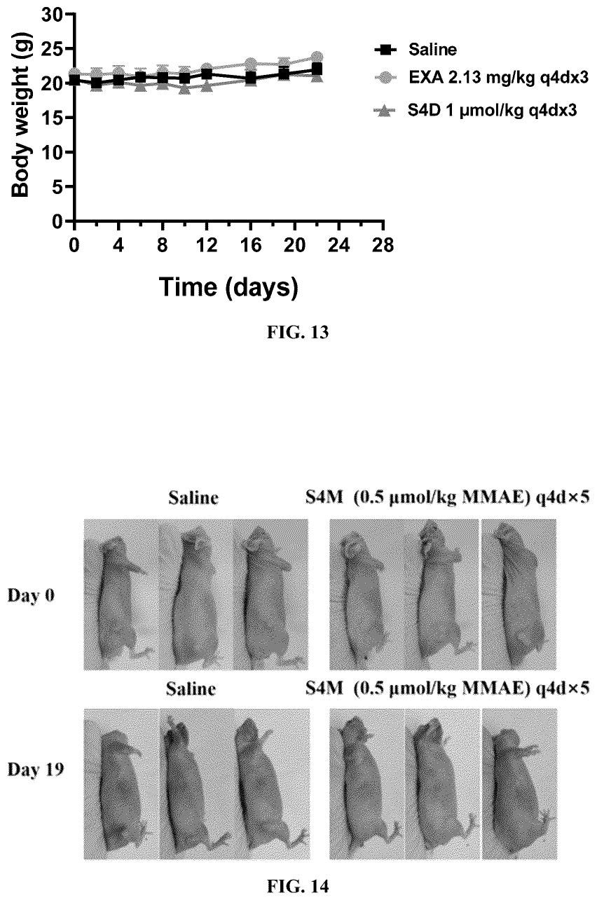

The results are shown in FIG. 12, indicating that at a dosage of 1 μmol/kg, the tumor in mice was effectively suppressed, achieving an inhibition rate of over 92%, which was significantly better than the effect of Exatecan. Mouse body weight was also monitored, and the results are shown in FIG. 13. There was no significant difference in body weight between the S4D group and the saline group (control group) or the Exatecan group, indicating that the ApDC S4D with a drug-loading ratio of 4 has good safety at this dosage.

### Example 15: The Effect of S4M on Tumor Suppression of Triple-Negative Breast Cancer PDX Model

Six mice bearing triple-negative breast cancer PDX models with tumor volumes of 50-150 mm³ were randomly divided into two groups. Treatments were administered every four days, with mice receiving either saline or the PTK7-targeted ApDC S4M at a drug-loading ratio of 4 (0.5 μmol/kg equivalent MMAE), for a total of six injections. Mouse body weights, tumor lengths (a), and tumor widths (b) were measured and recorded for each two days. The tumor volume (V) was calculated using the formula: V=(a×b²)/2. The experiment was terminated when tumor volume exceeded 1500 mm³ or body weight decreased by more than 15%, at which point euthanasia was performed.

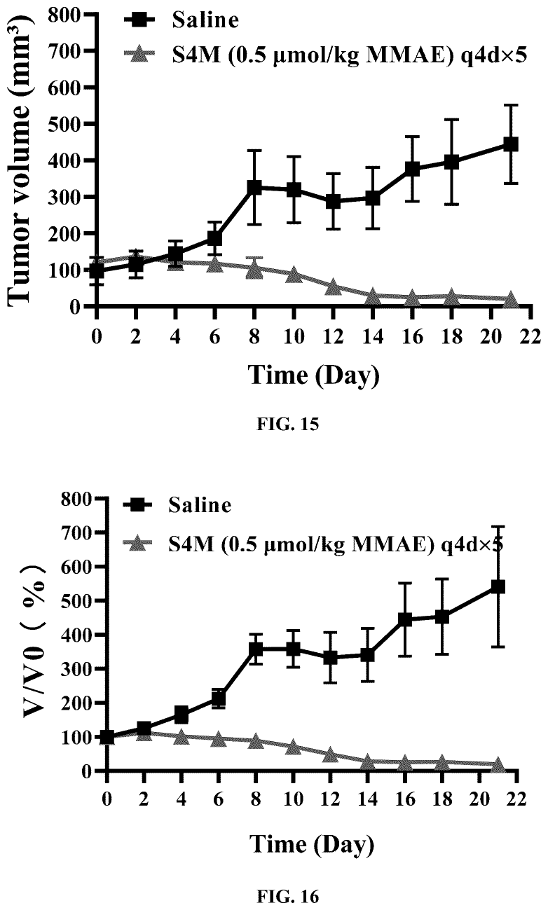

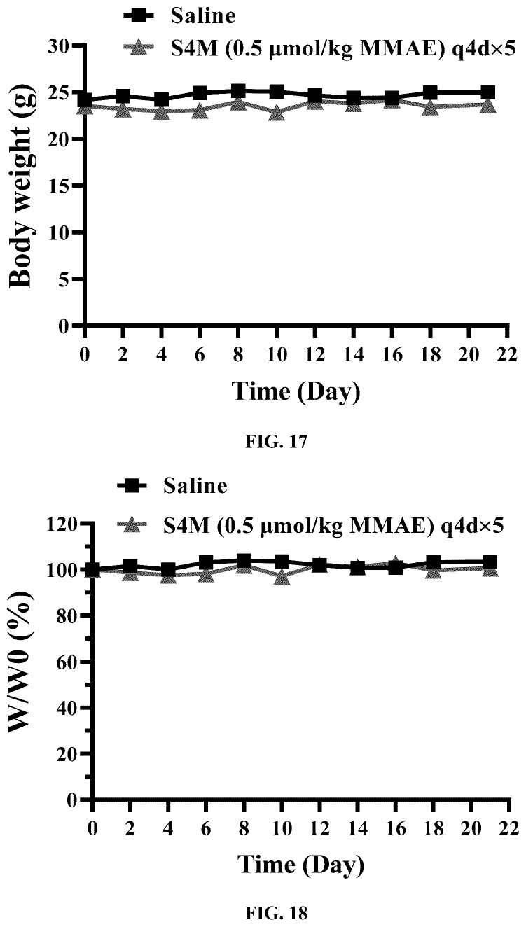

The results are shown in FIGS. 14-16, indicating that the tumors in mice were effectively suppressed, achieving an inhibition rate of over 95.4%. Mouse body weight was also monitored, and the results are shown in FIGS. 17-18. There was no significant difference in body weight between the S4M group and the saline group, indicating that the PTK7-targeted ApDC S4M has good biological safety at this dosage.

### Example 16: The Tumor Suppressive Effect of High Drug-Loading Ratio ApDCs on Lung Cancer NCI-H1975 Cell Transplantation Tumor

Eighteen mice with subcutaneous tumors of the lung cancer cell line NCI-H1975, with tumor volumes between 150-200 mm³, were randomly divided into six groups. Treatments were administered every four days (for a total of three injections) or seven days (for a total of two injections), consisting of tail vein injections of saline, the clinical frontline chemotherapy drug Paclitaxel (10 mg/kg), and PTK7-targeted ApDCs S2M and S4M with drug-loading ratios of 2 and 4, respectively, at doses of 1.0 μmol/kg and 0.5 μmol/kg equivalent of MMAE. Mouse body weight, tumor length (a), and tumor width (b) were measured and recorded for each two days. The tumor volume (V) was calculated using the formula: V=(a×b²)/2. The experiment was terminated when the tumor volume exceeded 1500 mm³ or if body weight was reduced by more than 15%, at which point euthanasia was performed.

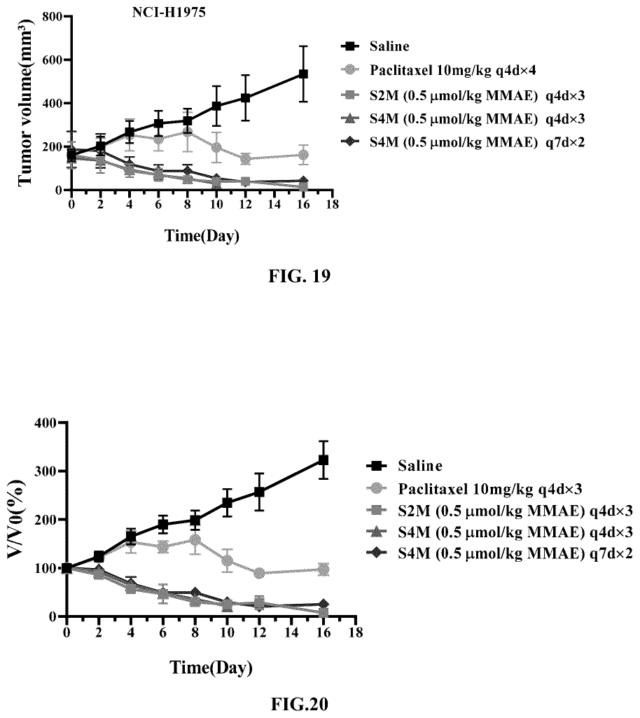

The results are shown in FIGS. 19-20. Compared to the frontline chemotherapy drug Paclitaxel, the different drug-loading ratio PTK7 ApDCs effectively inhibited tumor growth in mice at a dose of 0.5 μmol/kg equivalent MMAE. Moreover, the 0.5 μmol/kg equivalent S4M achieved effects comparable to the 1.0 μmol/kg equivalent S2M, with an inhibition rate exceeding 90%. It can also be observed that the ApDCs S2M and S4M exhibited similar good tumor-suppressive effects at the same MMAE dosage. Therefore, for the Maytansine analog microtubule inhibitor MMAE, S4M does not demonstrate an advantage over S2M. However, as noted in Example 13, S4D exhibits significant advantages compared to SD, S2D, S3D, S6D, and S8D, indicating that the structure of S4D is better suited for the DNA topoisomerase I inhibitor Dxd; nonetheless, S4M does not outperform S2M.

### Example 17: The Effect of S4M Concentration on Tumor Suppression in Colon Cancer HT-29 Cell Transplantation Tumor

Fifteen mice bearing human colon cancer HT-29 tumors, with tumor volumes ranging from 50 to 150 mm³, were randomly divided into five groups. Treatments were administered every four days, consisting of tail vein injections of saline, MMAE (0.5 μmol/kg), and the PTK7-targeted ApDC S4M with a drug-loading ratio of 4 at doses of 0.125, 0.25, and 0.5 μmol/kg equivalent of MMAE, for a total of three injections. Mouse body weight, tumor length (a), and tumor width (b) were measured and recorded for each two days. The tumor volume (V) was calculated using the formula: V=(a×b²)/2. The experiment was terminated when the tumor volume exceeded 1500 mm³ or if body weight was reduced by more than 15%, at which point euthanasia was performed.

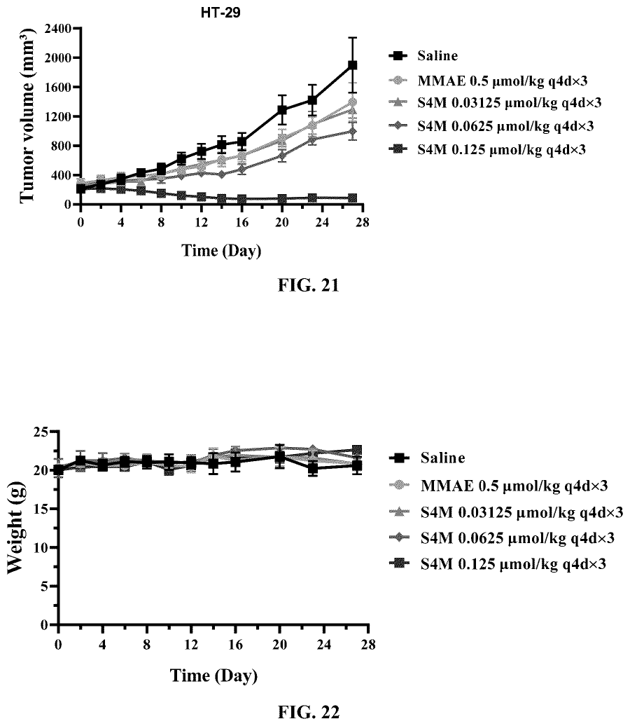

The results are shown in FIG. 21. The tumors in mice were effectively suppressed, and at the same MMAE dosage, the efficacy of the ApDC S4M with a drug-loading ratio of 4 was significantly enhanced. The efficacy of S4M with a drug-loading ratio of 4 demonstrated dose dependence, with efficacy increasing in the range of 0.125-0.5 μmol/kg equivalent MMAE dosing as the dose increased. At a dosage of 0.5 μmol/kg equivalent MMAE, the ApDC S4M (0.125 μmol/kg) achieved an inhibition rate of over 95.5%.

Mouse body weight was also monitored, and the results are shown in FIG. 22. There was no significant difference in body weight between the different doses of the ApDC S4M with a drug-loading ratio of 4 and the saline control group, indicating that the high drug-loading ratio ApDCs have good biological safety at these dosages.

### Example 18: The Tumor Suppressive Effect of High Drug-Loading Ratio ApDCs on Ovarian Cancer OVCAR3 Cell Transplantation Tumor

Twelve mice bearing human ovarian cancer OVCAR3 tumors, with tumor volumes ranging from 100 to 200 mm³, were randomly divided into four groups. Treatments were administered every four days, consisting of tail vein injections of saline, MMAE (0.5 μmol/kg), the PTK7-targeted ApDC S2M at 0.25 μmol/kg equivalent (with a drug-loading ratio of 2), and the PTK7-targeted ApDC S4M at 0.125 μmol/kg equivalent (with a drug-loading ratio of 4), for a total of three injections. Mouse body weight, tumor length (a), and tumor width (b) were measured and recorded for each two days. The tumor volume (V) was calculated using the formula: V=(a×b²)/2. The experiment was terminated when the tumor volume exceeded 1500 mm³ or if body weight was reduced by more than 15%, at which point euthanasia was performed.

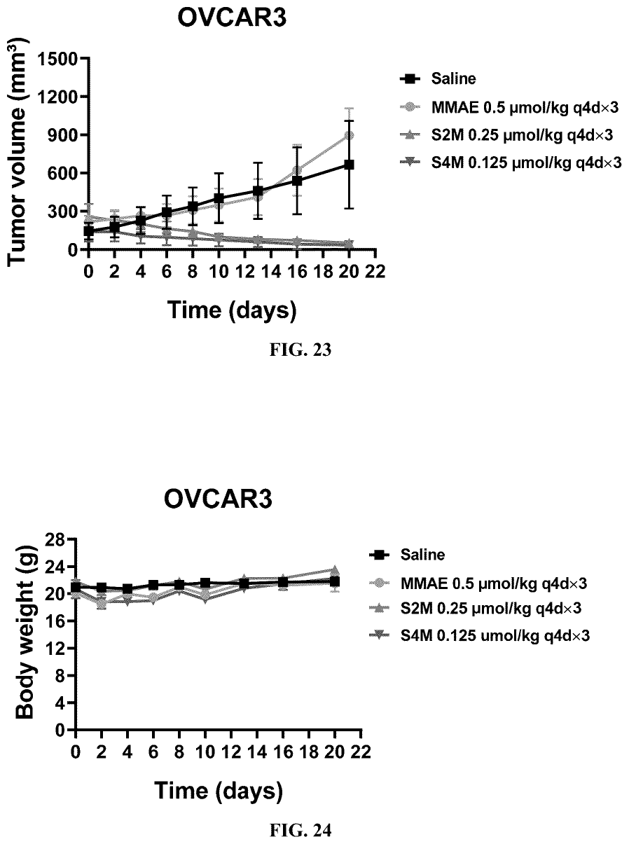

The results are shown in FIG. 23. Compared to the MMAE drug group and the control group, the tumors in the S2M and S4M groups were effectively suppressed, with inhibition rates of 92.2% and 94.8%, respectively. This indicates that at the same MMAE dosage, the ApDC S4M with a drug-loading ratio of 4 has a more significant efficacy.

Mouse body weight was also monitored, and the results are shown in FIG. 24. The weights of mice in the different drug-loading ratio ApDC groups displayed slight fluctuations after treatment, but there were no significant differences compared to the saline control group, indicating that the high drug-loading ratio ApDCs have good biological safety at this dosage.

### Example 19: The Inhibitory Effect of High Drug-Loading Ratio ApDCs on Lung Cancer NCI-H1975 Cell Transplantation Tumor

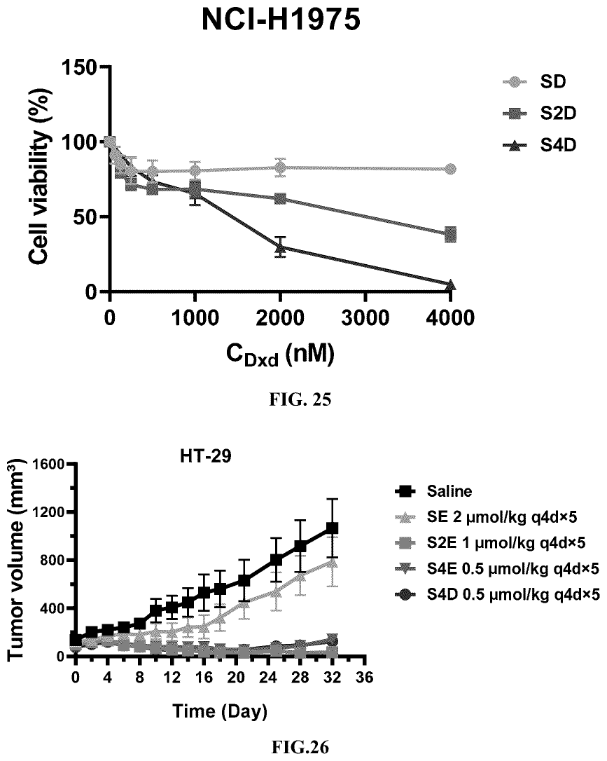

NCI-H1975 cells were inoculated into a 96-well plate (4000 cells per well) and cultured for 24 hours at 37 degrees with 5% CO₂. Different concentrations of PTK7 ApDCs SD (Sgc8c-Dxd), S2D (Sgc8c-2Dxd), and S4D (Sgc8c-4Dxd) with varying drug-loading ratios were added. After 24 hours of culture under the same conditions, the drug-containing medium was discarded, and a drug-free medium was added to continue the culture for another 108 hours. After a total of 120 hours, the drug's inhibitory effect was assessed using the MTS reagent kit, and the results are shown in FIG. 25. As shown in FIG. 25, the inhibitory effects of the ApDCs SD, S2D, and S4D on non-small cell lung cancer NCI-H1975 cells gradually increased with higher drug loading, with the order of inhibitory activity being S4D>S2D>SD; S4D showed more than a 4-fold reduction in IC50 compared to SD. This indicates that for different drugs, multi-drug loading enhances drug toxicity and increases efficacy.

### Example 20: Comparative Efficacy of High Drug-Loading Ratio ApDCs

Twenty mice bearing human colon cancer HT29 tumors, with tumor volumes ranging from 50 to 150 mm³, were randomly divided into five groups. Treatments were administered every four days, consisting of tail vein injections of saline, SE (2 μmol/kg), S2E (1 μmol/kg), S4E (0.5 μmol/kg), and S4D (0.5 μmol/kg), all equivalent to 2 μmol/kg, for a total of five injections. Mouse body weight, tumor length (a), and tumor width (b) were measured and recorded for each two days. The tumor volume (V) was calculated using the formula: V=(a×b²)/2. The experiment was terminated when the tumor volume exceeded 1500 mm³ or if body weight was reduced by more than 15%, at which point euthanasia was performed.

The results are shown in FIG. 26. The tumors in the mice were effectively suppressed, and at the same EXA dosage, the ApDCs S2E and S4E showed similar good tumor-suppressive effects, with inhibition rates exceeding 96.8%. This suggests that for the DNA topoisomerase I inhibitor Exatecan, S4E does not demonstrate an advantage over S2E. However, as noted in Example 13, S4D shows significant advantages compared to SD, S2D, S3D, S6D, and S8D, indicating that the structure of S4D is better suited for the DNA topoisomerase I inhibitor Dxd.

It can also be observed from FIG. 26 that there is no significant difference between S4D and S4E at a dosage of 0.5 μmol/kg. This indicates that a low concentration of S4D can already achieve the effects of S4E, and it also suggests that the structure of S4D is particularly suitable for the DNA topoisomerase I inhibitor Dxd, significantly enhancing its antitumor effects.

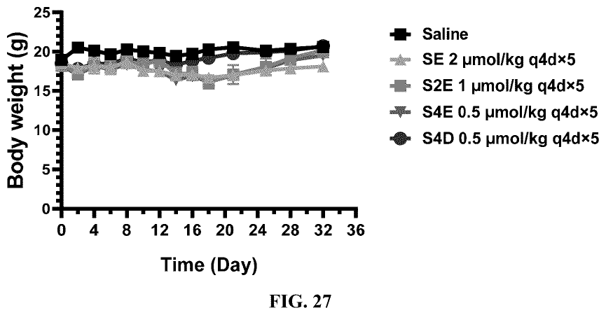

Mouse body weight was monitored, and the results are shown in FIG. 27. The mouse body weights in the different drug-loading ratio ApDC groups decreased after treatment, but after stopping the medication, the mouse weights rapidly returned to levels consistent with the saline control group, indicating that the high drug-loading ratio ApDCs are tolerable at this dosage.

### Example 21: ApDC with a Drug-Loading Ratio of 2 which Drug Conjugated at Both Ends of Aptamer

This example investigates the differences between attaching drugs to both ends of a aptamer versus only attaching them to the 5′ end.

The MMAE drug is conjugated to both ends of the aptamer sgc8c (PTK7), resulting in a ApDC MSM, which has a drug-loading ratio of 2.

The construction process of MSM is represented by the following reaction equation:

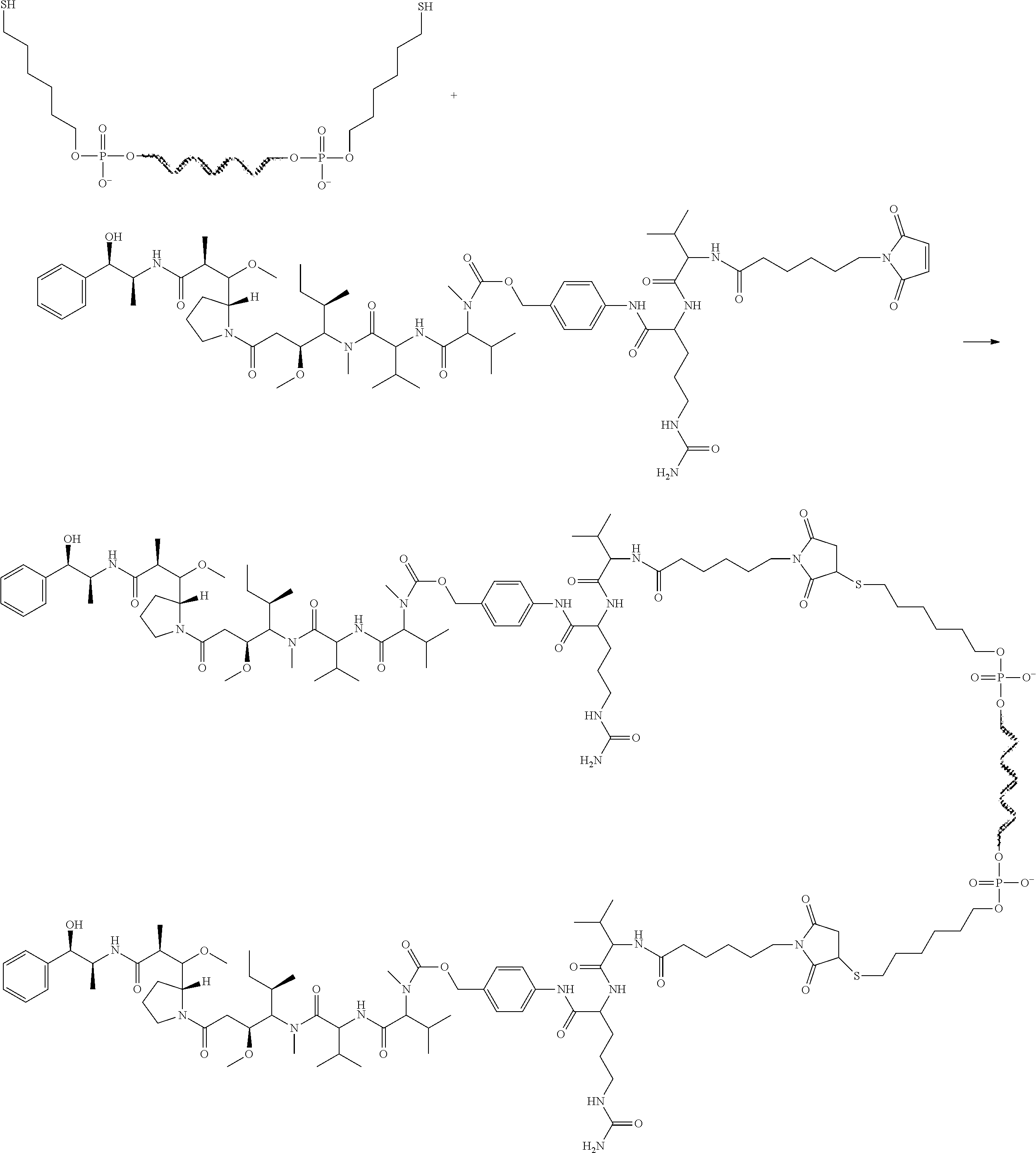

The construction process is as follows: using a solid-phase synthesizer, sgc8c modified with thiol groups at both ends is synthesized. After purification, it is reacted with vcMMAE to construct MSM, under the reaction conditions referenced in Example 2. After HPLC purification, MSM is obtained, with one MMAE drug attached to each end.

MSM is compared with S2M, which is constructed based on a bifunctional linker (attached at the 5′ end) from Example 2, regarding stability in mouse plasma. Both drugs are incubated with mouse plasma, and samples are taken at 0.5 h, 1 h, and 3 h for analysis using gel electrophoresis and liquid chromatography-mass spectrometry. The results are shown in the figure; MSM rapidly degrades in mouse plasma, showing degradation bands, while S2M shows no significant degradation over the 3-hour period.

Further characterization of metabolic products was performed using liquid chromatography-mass spectrometry (LC-MS), and the results indicate that the dipeptide linker in vcMMAE of MSM degrades in mouse plasma, leading to drug degradation, while S2M shows no significant dipeptide linker breakdown within the 3 hours, suggesting that the MSM constructed with connections at both ends suffers from poor stability and is clearly less effective than S2M. This also indicates that the bifunctional linker-based drug with a connection at the 5′ end, such as the multipharmaceutical-loaded ApDC, can significantly improve drug stability and provide better linker stability, thus preventing the release of drugs at non-target sites that could cause toxicity.

We simultaneously compared the cytotoxicity of MSM and S2M in HT-29 cells, and the results showed that the IC50 of S2M was more than 10 times lower than that of MSM, indicating that multiple payloads (ApDC) with the bifurcated linker conjugate can significantly enhance potency of drugs.

Although the present invention has been disclosed as above, it is not limited to this. Any technician skilled in the art can make various changes and modifications without departing from the spirit and scope of the present invention, and therefore, the protection scope of the present invention should be defined by the claims.

1. A high drug-loading aptamer-drug conjugates comprising:
an aptamer conjugated with a drug;
3′ and/or 5′ end of the aptamer being conjugated with one or more dendritic phosphoramidite monomer, wherein the dendritic phosphoramidite monomer is selected any one or more of Phosphoramidite 1, Phosphoramidite 2, or Phosphoramidite 3; wherein the structures of Phosphoramidite 1, Phosphoramidite 2, and Phosphoramidite 3 are as follows: 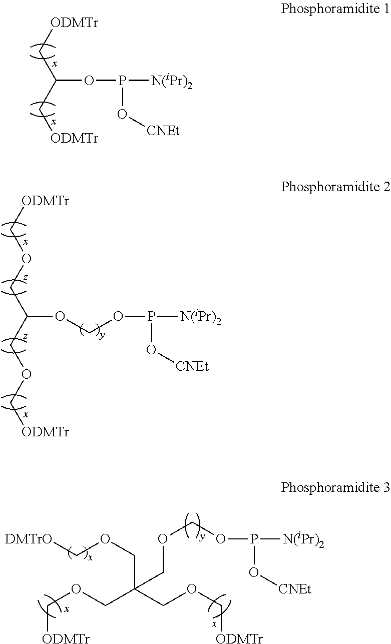
and wherein x is an integer from 1 to n; y is an integer from 1 to n; z is an integer from 1 to n; DMTr is a 4,4′-dimethoxytrityl protecting group, ⁱPr is isopropyl, and CNEt is cyanoethyl.

2. The aptamer-drug conjugates according to claim 1, further comprising one or more reactive groups that are conjugated to the 3′ and/or 5′ end of the aptamer, and wherein reactive groups is selected one or two from thiol, amino, DBCO, azide, and maleimide.

3. The aptamer-drug conjugates according to claim 2, wherein the reactive groups are conjugated to the 3′ end of the aptamer. These reactive groups are conjugated with the dendritic phosphoramidite monomer.

4. The aptamer-drug conjugates according to claim 1, wherein the aptamer can target one or more of the following antigens: CD5, CD19, CD20, CD25, CD37, CD30, CD33, CD45, CAMPATH-1, HLA-DR, CEA, TAG-72, EpCAM, MUC1, MUC15, folate-binding protein, A33, G250, PSMA, ferritin, GD2, GD3, GM2, Leg, CA-125, CA19-9, epidermal growth factor, p185HER2, IL-2 receptor, tenascin, a metalloproteinase, endosialin, vascular endothelial growth factor, avB3, WT1, LMP2, HPV E6, HPV E7, EGFR, EGFRVIII, Her-2/neu, MAGE A3, p53 nonmutant, NY-ESO-1, MelanA/MART1, Ras mutant, gp100, p53 mutant, PR1, bcr-abl, tyrosinase, survivin, PSA, hTERT, a sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, MSLN, PSCA, MAGE AI, MAGE-A3, sLe, CYP1B1, PLAVI, GM3, BORIS, Tn, GloboH, ETV6-AML, NY-BR-1, RGS5, SART3, STn, carbonic anhydrase IX, PAX5, OY-TESL sperm protein 17, LCK, HMWMAA, AKAP-4, 55X2, XAGE 1, B7H3, legumain, Tie 3, Page 4, VEGFR2, MAD-CT-1, PDGFR-B, MAD-CT-2, ROR2, CMET, HER3, EPCAM, CA6, NAPI2B, TROP2, CLDN18.2, FAP, RON, LY6E, FRA, DLL3, PTK7, LIV1, ROR1, Fos-related antigen 1, VEGFR, endoglin, PD-L1, CD204, Nectin-4, CD206, CD301, VTCN1, CD71, and VISTA.

5. The aptamer-drug conjugates according to claim 1, wherein that drug comprises one or more of auristatin E (MMAE), deruxtecan (Dxd), exatecan, or a derivative that has the same or similar core structure as MMAE, Dxd, or exatecan.

6. The aptamer-drug conjugates according to claim 5, wherein the aptamer further comprises one or more number of conjugation sites, and the conjugation sites are 2, 3, 4, 6, or 8; and wherein the drug further includes a linker, and one end of the linker is conjugated to the 5′ end of the aptamer, another end of the linker is conjugated to the drug.

7. The aptamer-drug conjugates according to claim 6, wherein in that when the drug is Dxd, the number of conjugation sites of the aptamer is 4.

8. The aptamer-drug conjugates according to claim 6, wherein when the drug is exatecan, the number of conjugation sites of aptamer are 2 or 4.

9. The aptamer-drug conjugates according to claim 6, wherein when the drug is auristatin E, the number of conjugation sites of aptamer is 2 or 4.

10. The aptamer-drug conjugates according to claim 7, wherein when the drug is Dxd, the aptamer-drug conjugates is the structural formula:

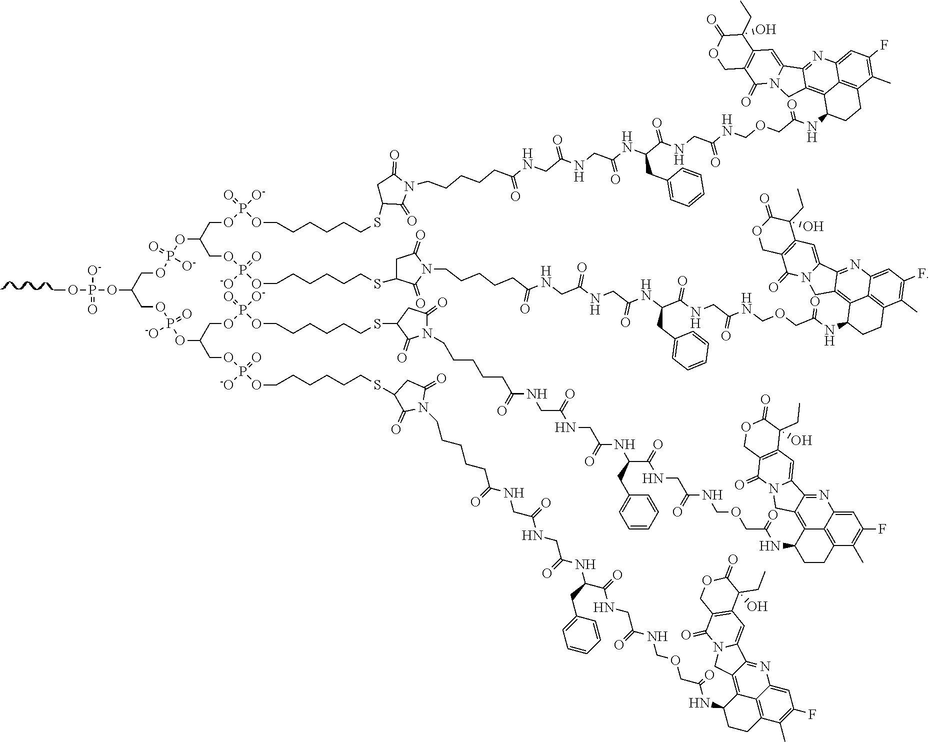

11. The aptamer-drug conjugates according to claim 8, wherein when the drug is exatecan, the aptamer-drug conjugates is the structural formula as below: 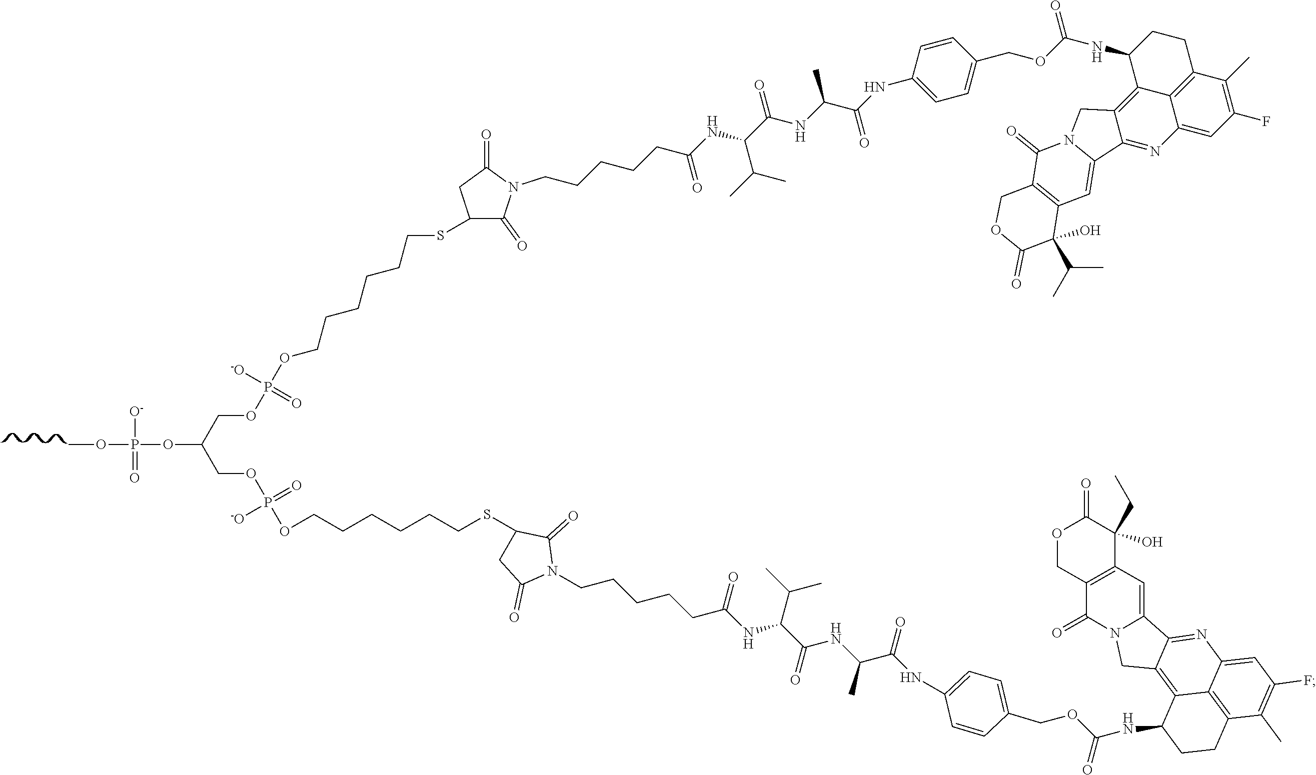
or:

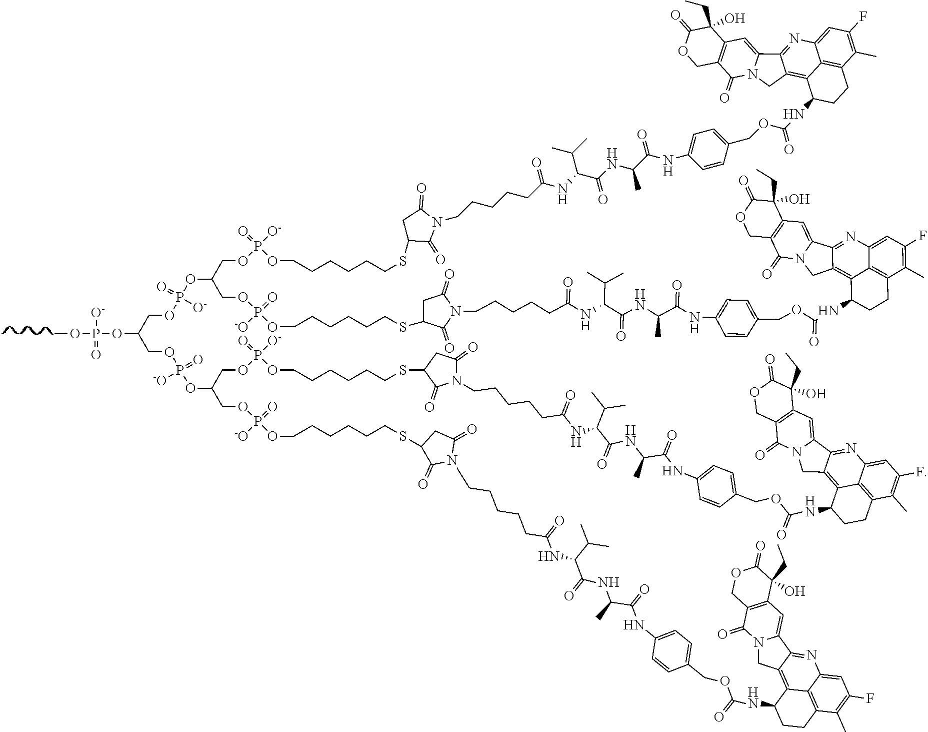

12. The aptamer-drug conjugates according to claim 9, wherein when the drug is auristatin E, the aptamer-drug conjugates is the structural formula as below: 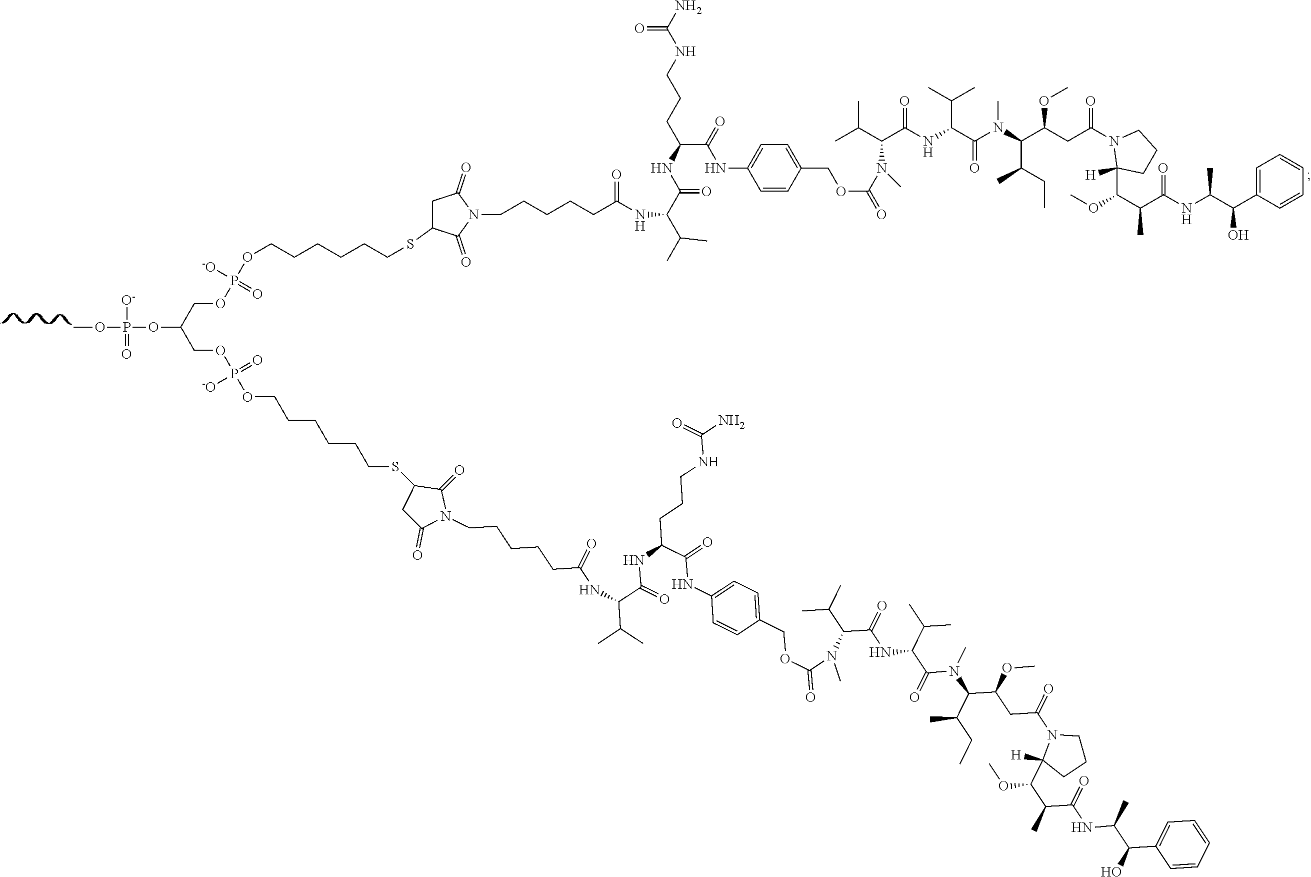
or:

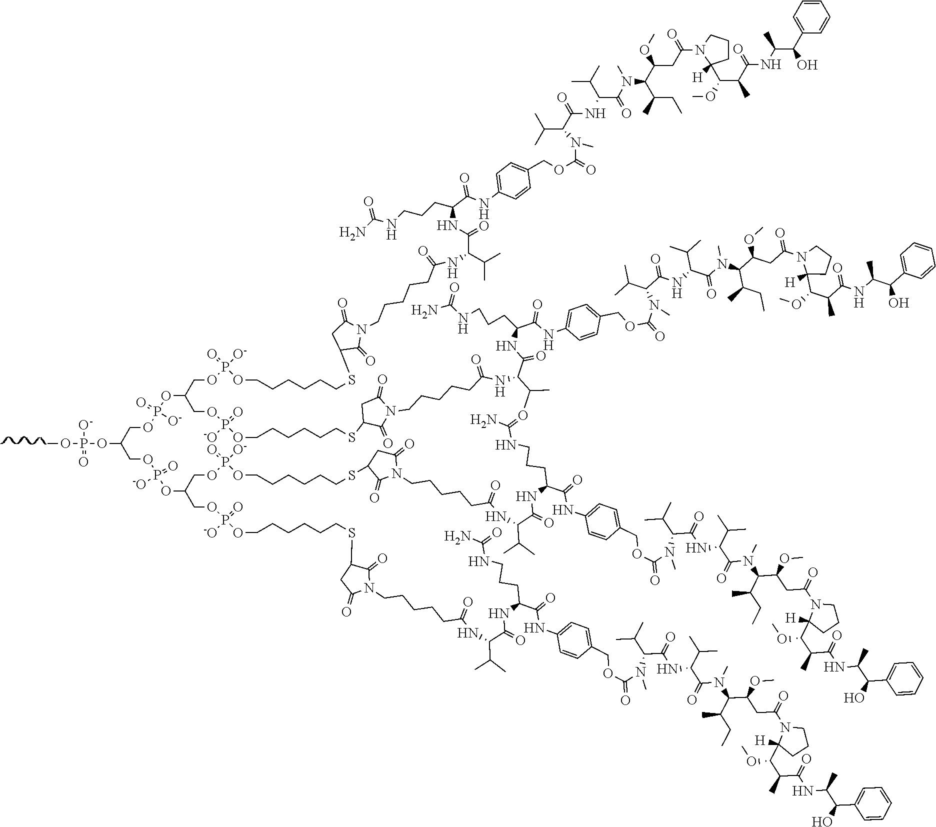

13. A method for enhancing the antitumor effect in individuals with colorectal cancer, comprising: injecting the individual with an aptamer-drug conjugates, wherein that the aptamer-drug conjugate is S4D, with the structural formula being:

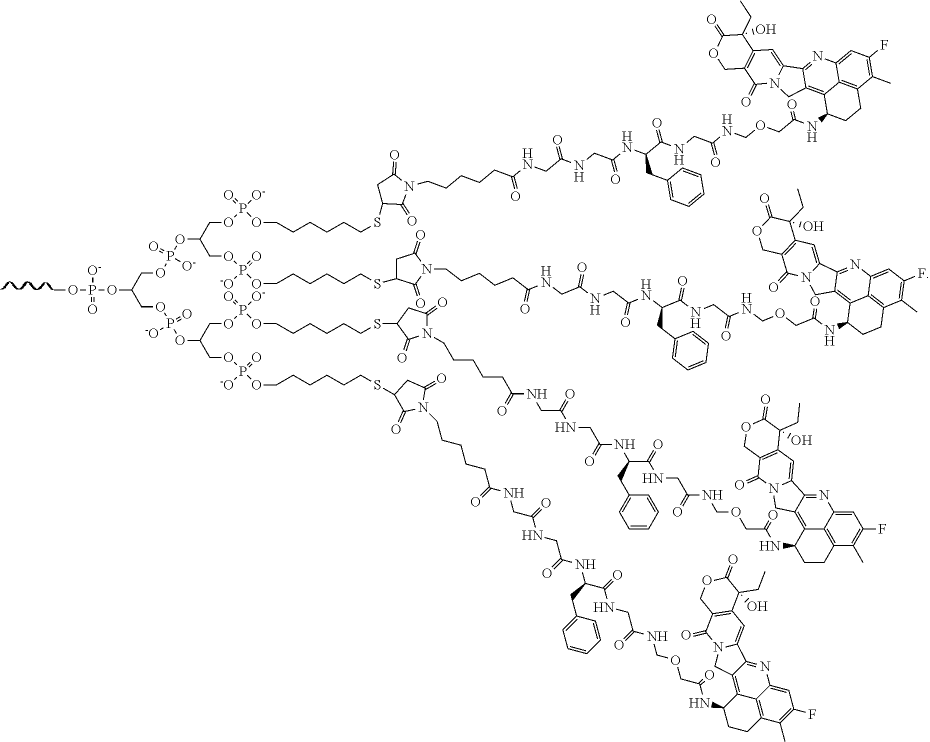

14. A method for enhancing the efficacy of the DNA topoisomerase I inhibitor Dxd to exceed the in vivo antitumor effect of exatecan, characterized in that an aptamer-drug conjugate is injected into the individual, wherein the aptamer-drug conjugates is SAD, with the structural formula being: [chemical structure]


SEQUENCE LISTING (2 sequences)

SEQ ID NO: 1 - DNA, 41 bp, synthetic construct
  source 1..41: mol_type = other DNA
atctaactgctgcgccgccgggaaaatactgtacggttaga

SEQ ID NO: 2 - DNA, 50 bp, synthetic construct
  source 1..50: mol_type = other DNA
atcaggctggatggtagctcggtcggggtgggtgggttggcaagtctgat